SEQUENCE LISTING (18 sequences)

SEQ ID NO: 1 - DNA, 6465 bp, Hypsibius dujardini
cgatcccgcgaaattaatacgactcactataggggaattgtgagcggataacaattcccctctagaaataattttgtttaactttaagaaggagatataccatgggcagcagcatggctaccaaggaatcaaaatatgaacgggtggagaaagtgaacgttgatgctgacggggctacactggtcaagaacatcggcgaggatcgggggaaagaagaccctggcatgaactttcaagacaagcgccctgcaaatcttgtcccaggcgccccggccggtgttatcccgaaccgcatcgaatcgctgccgactgatcgcgcgggccaacgtctgcgtgaacacctgagtgagtcggaacggctccgtgtgtcgcgttcgtccaccagtagtaaatcgagttcgttcgtcgaaccctccctgaaatatcgcggggaaatcggaccgatcggtaaaaacggtgagttcgtggcgagcagcaatcggcaaaatagtagttccaacgtcagcagtagtgataacagcgagcgcgccagcccagcctctcgcaattcgaatccaggcatgaacaacggaatgacaacacaacgcactaccgtgatcaccgaatcgagcgtgcagggcctgggtgcgcagcggaccgttccgattcagccgcatcagcagcgcgaagatcatgaagtgattacgcatgaatctcatgctcgcgctcccgagactacggtggtgacaatcccgacaacgcgctttgaaagcgcgcagctggaatctcgccgcgacggccgcacctataccgaggataaagaactcaccattccggcgccggtggtggcgccgcagattcatgcgcaccagcaagtgaatatgtcgggcgggacgtcggctacaattcacgccactacggatcttcatttagcgtcagaggcgcagatcaatgacatgggcccggaggagtatgagcgctatcgggcgaaagtagaggcgctggctcgcattcatgaagatgaaacaagccgtaaggcggcagcatatcgtaacgccgtcgaggcagacgcagaattgattcgccagactctggaacggcagcatatgcgggatatcgagttccgcaaggatctggtcgagagttccgttgatcgtcaacagcaagagatccgcttagaagccgagtatgcgatgcgtgccctggagcaggagcgtgttaatgcgcgcgcggcgctcgatcaggcgatggcgagtaccaacattgatgtcaacattgattcggcaatcggcacgacgcactcgcagggacgcgttacaaccacctccgagtcacggacatcacaagcccgcggtccagcaaccgcagcagtcatctaagagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatct

SEQ ID NO: 2 - DNA, 5904 bp, Hypsibius dujardini
cgatcccgcgaaattaatacgactcactataggggaattgtgagcggataacaattcccctctagaaataattttgtttaactttaagaaggagatataccatgggcagcagcatgtcagggcgtaacgtggagtcccatatggagcgcaacgaaaaagtggtagtgaataactccggtcatgcggacgtgaaaaaacagcaacaacaggttgaacatacggagttcacgcatacagaagtaaaagccccgctcatccacccagcccctccgattatttcgactggcgccgcgggcttagcggaggaaattgtgggccagggttttactgcgtcagcagcgcgtatctcaggtggcactgccgaagtgcatctgcagccgtcagcggcgatgacagaagaagctcgccgtgatcaggaacgttatcggcaggagcaggaaagtattgcgaaacaacaggaacgggaaatggaaaagaagaccgaagcgtaccgcaagacggcggaggcggaagctgaaaaaattcgtaaagaactggaaaaacaacatgcgcgcgatgtcgaattccgtaaagatctgatcgaatccacgatcgatcgtcagaaacgtgaagtggatctggaagcgaaaatggctaaacgcgagttagatcgtgaaggtcagctggctaaagaagccctggaacgctctcggttagccacgaacgtcgaagttaacttcgattcggcagccgggcatacagtcagtggagggaccactgttagcactagcgataagatggaaattaaacgcaactaagagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatct

SEQ ID NO: 3 - DNA, 5904 bp, Hypsibius dujardini
cgatcccgcgaaattaatacgactcactataggggaattgtgagcggataacaattcccctctagaaataattttgtttaactttaagaaggagatataccatgggcagcagcatggaggccatgaatatgaatatcccccgcgatgccatgtttgttccgccaccggaatctgagcaaaatgggtatcatgagaagtcagaagttcagcaaacaagttatatgcagagtcaagtcaaagtgccacattataatttcccgacaccatattttacgacttccttttctgcgcaagagctgctgggcgaagggtttcaagcctcaatttcccgtatttcagccgttacggaagacatgcagagcatggaaatcccggagttcgttgaagaggcccgccgtgattacgcagccaaaacacgtgaaaatgagatgctggggcaacaatatgaaaaagagctggaacgtaagtccgaagcctaccgcaaacatcaggaagtagaggccgacaaaatccgcaaagaacttgaaaaacagcatatgcgtgatattgaatttcggaaagaaattgcagaactggcgattgagaaccaaaaacgtatgatcgatcttgaatgccgctatgcaaaaaaagacatggaccgggaacgcacaaaagttcgtatgatgctggagcaacagaaattccatagtgatatccaggtaaatctggattcttctgcggctgggaccgagagcggaggtcatgtagtgagccagtctgaaaagttcaccgaacgtaaccgcgagatgaaacgctaagagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatct

SEQ ID NO: 4 - protein, 224 aa, Hypsibius dujardini
MSNYQQESSYQYSDRSNNGQQQEQQEKKEVEHSSYTHTDVKVNMPNLIAPFISSSAGLAQELVGEGFQASVSRITGASGELTVIDTEAETEEARRDMEAKAREQELLSRQFEKELERKTEAYRKQQEVETEKIRKELEKQHLRDVEFRKELMEQTIENQKRQIDLEARYAKKELERERNKVKRVLERSKFHTDIQVNMEAAAGSTHSGSSSVAVSESEKFQTNN

SEQ ID NO: 5 - DNA, 675 bp, Hypsibius dujardini
atgtctaactaccagcaagaatccagctatcagtacagcgaccggagcaacaatggtcaacagcaagagcaacaggagaagaaggaggtcgaacactccagctatacccacaccgacgtcaaagtgaacatgcccaacttaatcgcacctttcatcagctcttccgcgggtttggctcaagaactggtcggggaaggtttccaggcgtccgtctctcgcatcaccggcgcatccggggagctcaccgtcatcgacaccgaggccgaaaccgaggaggcacgtcgggacatggaagccaaggcccgcgagcaggagctcctgtcgcgacagtttgagaaggagctggagcgaaagaccgaagcgtatcgcaagcagcaagaagttgagaccgagaagatccggaaggaacttgagaagcaacatctgcgggatgtcgagttccgcaaggagttgatggagcagaccatcgagaatcagaagcgtcagatcgacctggaggcacgctatgccaagaaggagcttgagcgggaacggaacaaggtcaagcgtgtgctggaacgctccaaattccacaccgatatccaggtcaacatggaagccgctgcgggttcaactcattccggatcatccagcgttgccgtgtcggagtcggaaaagttccagaccaacaactga

SEQ ID NO: 6 - DNA, 675 bp, Artificial
  Synthetic
atgagtaactatcagcaagaatccagctatcaatatagcgatcgcagtaataatggccagcaacaagaacagcaagaaaagaaggaagtagaacactcgagttacacgcacactgacgtaaaagtaaatatgccgaacttgattgccccttttatttcatcttctgcgggtctggcgcaggaattggtcggcgagggtttccaagcaagcgtatctcgtatcactggcgcatcgggcgaattaactgtgattgatactgaagcagaaaccgaggaagctcgccgtgacatggaagccaaggcacgtgaacaggagctcctgagtcgtcagtttgaaaaagaactggaacgtaaaaccgaggcctatcgcaaacagcaggaagttgaaactgaaaaaatccgcaaagaattagaaaaacagcatctgcgcgatgtggagttccgtaaagaactcatggaacagactattgaaaaccaaaaacgccagattgacctcgaagcgcgctacgctaagaaagagctcgagcgtgagcggaataaagtcaaacgcgttctggaacgtagcaaatttcatacagacatccaggtaaacatggaagcggctgcgggttctactcacagcggctcaagctctgtggctgttagcgaaagtgaaaagttccagaccaataactaa

SEQ ID NO: 7 - protein, 414 aa, Hypsibius dujardini
MATKESKYERVEKVNVDADGATLVKNIGEDRGKEDPGMNFQDKRPANLVPGAPAGVIPNRIESLPTDRAGQRLREHLSESERLRVSRSSTSSKSSSFVEPSLKYRGEIGPIGKNGEFVASSNRQNSSSNVSSSDNSERASPASRNSNPGMNNGMTTQRTTVITESSVQGLGAQRTVPIQPHQQREDHEVITHESHARAPETTVVTIPTTRFESAQLESRRDGRTYTEDKELTIPAPVVAPQIHAHQQVNMSGGTSATIHATTDLHLASEAQINDMGPEEYERYRAKVEALARIHEDETSRKAAAYRNAVEADAELIRQTLERQHMRDIEFRKDLVESSVDRQQQEIRLEAEYAMRALEQERVNARAALDQAMASTNIDVNIDSAIGTTHSQGRVTTTSESRTSQARGPATAAVI

SEQ ID NO: 8 - DNA, 1245 bp, Hypsibius dujardini
atggccaccaaggaatcaaagtatgaacgcgtggagaaggtcaacgtggacgcggatggcgccacactggtcaagaatatcggcgaggaccgcggcaaggaggatcccgggatgaatttccaggacaaacgcccggccaatttggtgcccggagcgccggccggagtcattcccaaccgtatcgagtcattgccgacggatcgtgccgggcaacgtctccgtgagcatctcagcgagagcgaacgccttcgcgtctcccgcagcagcaccagcagcaagtcttccagcttcgtggagcccagcctcaagtaccgcggtgagatcggaccgatcggaaaaaacggcgagttcgtcgcctcgtcgaaccgtcagaactccagcagcaacgtctcgtcctccgacaacagcgagcgtgcgtctccggcgtcccgcaactccaaccccgggatgaacaacggcatgacgacccaacgcaccacggtgatcacggagagctcggttcagggcctcggcgcgcaacgcaccgtcccgatccagccgcatcagcagcgcgaagaccacgaggtgatcacccacgaatcgcatgctcgagctccggaaacgaccgtggtgaccattcccacgacgcgcttcgagtccgcgcagctggagtcgcgccgcgacggcagaacctacaccgaggacaaggagctgaccattccggcgccggtggtcgcgccgcagatccacgcccaccagcaggttaacatgtccggaggaacttcggcgacgatccacgccacgaccgatttacatttggctagcgaggcacagatcaatgacatgggaccagaggagtatgagcgctaccgtgccaaggtggaagccttggctcgcatacatgaggacgaaacgtcgcgcaaagcggcggcctaccgtaatgccgttgaggccgatgcggaactcatccgccaaactctggagcgccaacacatgcgtgacattgaattccgcaaggatctagttgaaagctcggtggatcgtcaacagcaggagatccgcctggaggccgagtacgccatgcgagccctggaacaggaacgtgtcaatgctcgcgctgctcttgaccaagccatggcgtcgacgaacatcgacgtgaacattgactccgctatcggcacgacccattcccagggaagggttacgacgacctctgaaagccgaacaagtcaggcacgtggacccgccactgccgcagttatctga

SEQ ID NO: 9 - DNA, 1245 bp, Artificial
  Synthetic
atggctaccaaggaatcaaaatatgaacgggtggagaaagtgaacgttgatgctgacggggctacactggtcaagaacatcggcgaggatcgggggaaagaagaccctggcatgaactttcaagacaagcgccctgcaaatcttgtcccaggcgccccggccggtgttatcccgaaccgcatcgaatcgctgccgactgatcgcgcgggccaacgtctgcgtgaacacctgagtgagtcggaacggctccgtgtgtcgcgttcgtccaccagtagtaaatcgagttcgttcgtcgaaccctccctgaaatatcgcggggaaatcggaccgatcggtaaaaacggtgagttcgtggcgagcagcaatcggcaaaatagtagttccaacgtcagcagtagtgataacagcgagcgcgccagcccagcctctcgcaattcgaatccaggcatgaacaacggaatgacaacacaacgcactaccgtgatcaccgaatcgagcgtgcagggcctgggtgcgcagcggaccgttccgattcagccgcatcagcagcgcgaagatcatgaagtgattacgcatgaatctcatgctcgcgctcccgagactacggtggtgacaatcccgacaacgcgctttgaaagcgcgcagctggaatctcgccgcgacggccgcacctataccgaggataaagaactcaccattccggcgccggtggtggcgccgcagattcatgcgcaccagcaagtgaatatgtcgggcgggacgtcggctacaattcacgccactacggatcttcatttagcgtcagaggcgcagatcaatgacatgggcccggaggagtatgagcgctatcgggcgaaagtagaggcgctggctcgcattcatgaagatgaaacaagccgtaaggcggcagcatatcgtaacgccgtcgaggcagacgcagaattgattcgccagactctggaacggcagcatatgcgggatatcgagttccgcaaggatctggtcgagagttccgttgatcgtcaacagcaagagatccgcttagaagccgagtatgcgatgcgtgccctggagcaggagcgtgttaatgcgcgcgcggcgctcgatcaggcgatggcgagtaccaacattgatgtcaacattgattcggcaatcggcacgacgcactcgcagggacgcgttacaaccacctccgagtcacggacatcacaagcccgcggtccagcaaccgcagcagtcatctaa

SEQ ID NO: 10 - protein, 227 aa, Hypsibius dujardini
MSGRNVESHMERNEKVVVNNSGHADVKKQQQQVEHTEFTHTEVKAPLIHPAPPIISTGAAGLAEEIVGQGFTASAARISGGTAEVHLQPSAAMTEEARRDQERYRQEQESIAKQQEREMEKKTEAYRKTAEAEAEKIRKELEKQHARDVEFRKDLIESTIDRQKREVDLEAKMAKRELDREGQLAKEALERSRLATNVEVNFDSAAGHTVSGGTTVSTSDKMEIKRN

SEQ ID NO: 11 - DNA, 684 bp, Hypsibius dujardini
atgtctggacgtaacgtagaaagccacatggagcggaatgagaaggttgtggtcaacaattccggtcatgctgacgtgaagaagcaacagcaacaagtggagcacaccgaattcacccacaccgaggttaaagccccgttgatccatcccgcacctccgatcatctcaactggagccgccggactcgccgaggagattgtgggacaagggttcaccgcgagtgccgcgcgcatcagtggaggtaccgctgaagtacatctccagccttcggccgcaatgacggaagaggcccgtcgcgatcaagagcgctaccgccaggaacaggagtcgatcgccaagcagcaggaacgtgagatggaaaagaagactgaggcataccgcaagaccgccgaggcggaagctgagaagatccgaaaggagctggagaagcaacacgcgcgtgatgtcgagttccgtaaggatctcatcgagagcacgattgaccggcaaaaacgcgaggtcgatctggaagcgaaaatggccaagcgggaattggatcgtgaagggcagttggccaaggaggcgttggaacgatcacgtttggccaccaacgttgaggtcaatttcgacagtgcagctggtcataccgtgtccggtgggacgaccgtatccacttcggacaagatggaaatcaagcgaaactag

SEQ ID NO: 12 - DNA, 684 bp, Artificial
  Synthetic
atgtcagggcgtaacgtggagtcccatatggagcgcaacgaaaaagtggtagtgaataactccggtcatgcggacgtgaaaaaacagcaacaacaggttgaacatacggagttcacgcatacagaagtaaaagccccgctcatccacccagcccctccgattatttcgactggcgccgcgggcttagcggaggaaattgtgggccagggttttactgcgtcagcagcgcgtatctcaggtggcactgccgaagtgcatctgcagccgtcagcggcgatgacagaagaagctcgccgtgatcaggaacgttatcggcaggagcaggaaagtattgcgaaacaacaggaacgggaaatggaaaagaagaccgaagcgtaccgcaagacggcggaggcggaagctgaaaaaattcgtaaagaactggaaaaacaacatgcgcgcgatgtcgaattccgtaaagatctgatcgaatccacgatcgatcgtcagaaacgtgaagtggatctggaagcgaaaatggctaaacgcgagttagatcgtgaaggtcagctggctaaagaagccctggaacgctctcggttagccacgaacgtcgaagttaacttcgattcggcagccgggcatacagtcagtggagggaccactgttagcactagcgataagatggaaattaaacgcaactaa

SEQ ID NO: 13 - protein, 227 aa, Hypsibius dujardini
MEAMNMNIPRDAMFVPPPESEQNGYHEKSEVQQTSYMQSQVKVPHYNFPTPYFTTSFSAQELLGEGFQASISRISAVTEDMQSMEIPEFVEEARRDYAAKTRENEMLGQQYEKELERKSEAYRKHQEVEADKIRKELEKQHMRDIEFRKEIAELAIENQKRMIDLECRYAKKDMDRERTKVRMMLEQQKFHSDIQVNLDSSAAGTESGGHVVSQSEKFTERNREMKR

SEQ ID NO: 14 - DNA, 684 bp, Hypsibius dujardini
atggaagcgatgaatatgaacattccccgggatgccatgtttgtgccgccgcccgagagcgagcaaaatgggtaccatgagaagagcgaggtccagcagaccagctacatgcagtcgcaggttaaggtgccccattataatttccccacgccctatttcaccacatctttctcggcacaagagctccttggcgagggatttcaggcatcgatttctcgcatcagcgccgtgacggaagatatgcaatcaatggagatccccgagttcgtcgaagaagcccgtcgcgattacgccgctaagaccagggagaatgagatgctggggcagcagtacgagaaggagttggagcggaaatcggaggcgtaccgaaagcatcaggaagtggaggccgacaagatccgtaaagaactggagaagcagcacatgcgcgacattgagttccgcaaggagatcgccgagctggccatcgagaaccagaagcgcatgatcgacctcgaatgccgctatgccaaaaaggatatggaccgtgaacgcaccaaggttcgaatgatgctcgaacaacaaaagttccacagcgatatccaggtcaatctggattcgtcggctgccggcacggaatctggtggtcatgtggtgtcacagtccgagaagtttaccgaacgaaaccgcgagatgaagcgataa

SEQ ID NO: 15 - DNA, 684 bp, Artificial
  Synthetic
atggaggccatgaatatgaatatcccccgcgatgccatgtttgttccgccaccggaatctgagcaaaatgggtatcatgagaagtcagaagttcagcaaacaagttatatgcagagtcaagtcaaagtgccacattataatttcccgacaccatattttacgacttccttttctgcgcaagagctgctgggcgaagggtttcaagcctcaatttcccgtatttcagccgttacggaagacatgcagagcatggaaatcccggagttcgttgaagaggcccgccgtgattacgcagccaaaacacgtgaaaatgagatgctggggcaacaatatgaaaaagagctggaacgtaagtccgaagcctaccgcaaacatcaggaagtagaggccgacaaaatccgcaaagaacttgaaaaacagcatatgcgtgatattgaatttcggaaagaaattgcagaactggcgattgagaaccaaaaacgtatgatcgatcttgaatgccgctatgcaaaaaaagacatggaccgggaacgcacaaaagttcgtatgatgctggagcaacagaaattccatagtgatatccaggtaaatctggattcttctgcggctgggaccgagagcggaggtcatgtagtgagccagtctgaaaagttcaccgaacgtaaccgcgagatgaaacgctaa

SEQ ID NO: 16 - protein, 229 aa, Hypsibius dujardini
MSAEAMNMNMNQDAVFIPPPEGEQYERKEKQEIQQTSYLQSQVKVPLVNLPAPFFSTSFSAQEILGEGFQASISRISAVSEELSSIEIPELAEEARRDFAAKTREQEMLSANYQKEVERKTEAYRKQQEVEADKIRKELEKQHLRDVEFRKDIVEMAIENQKKMIDVESRYAKKDMDRERVKVRMMLEQQKFHSDIQVNLDSSAAGTETGGQVVSESQKFTERNRQIKQ

SEQ ID NO: 17 - protein, 690 aa, Hypsibius dujardini
ATGTCTGCTGAAGCGATGAACATGAACATGAACCAGGACGCCGTGTTTATTCCCCCGCCCGAGGGTGAGCAGTACGAGCGAAAGGAGAAGCAGGAGATCCAGCAGACCAGCTACCTGCAGTCCCAGGTCAAGGTGCCCCTTGTCAACCTCCCCGCTCCGTTCTTCAGCACTTCCTTTTCTGCCCAAGAAATTCTCGGCGAAGGTTTCCAGGCTTCGATTTCGCGCATCAGCGCCGTCTCGGAAGAGCTGTCGTCCATCGAGATTCCCGAACTGGCCGAAGAGGCCCGTCGCGACTTCGCTGCCAAAACCCGTGAGCAGGAGATGCTGTCAGCCAATTATCAGAAGGAAGTGGAGCGCAAGACCGAGGCCTACCGCAAGCAGCAGGAAGTCGAGGCCGACAAGATCCGCAAAGAACTGGAGAAGCAGCATCTGCGTGACGTTGAGTTCCGCAAGGACATTGTCGAGATGGCCATCGAGAACCAGAAGAAAATGATCGACGTGGAGAGCCGCTACGCCAAGAAGGACATGGACCGCGAACGCGTCAAGGTTCGAATGATGCTCGAGCAGCAAAAGTTCCACAGCGACATCCAGGTCAATCTGGATTCTTCGGCTGCTGGCACGGAAACTGGAGGTCAGGTGGTGTCGGAATCTCAAAAGTTCACCGAACGAAACCGCCAGATAAAGCAATAA

SEQ ID NO: 18 - DNA, 690 bp, Artificial
  Synthetic
atgtcagcggaagctatgaacatgaacatgaaccaggatgcagtttttattcctcctccggaaggcgaacagtatgaacgcaaagagaaacaagagattcaacagacgtcgtacctgcagagccaggtaaaggttccgctggtcaacctgcccgcacctttcttctcaacctccttctccgcgcaggagatccttggggaaggtttccaggctagtatttctcggatttccgccgttagtgaagaactgtcctctatcgaaattccggagctggcggaagaggcccgtcgtgactttgcggccaaaacgcgcgaacaggagatgctgtctgctaactatcaaaaagaagttgagcgcaaaactgaagcctatcggaagcagcaagaagtggaggcagacaaaattcggaaagagctggaaaagcagcatctgcgtgacgtggagttccgcaaagatattgtagagatggcaatcgaaaaccagaaaaaaatgattgatgtcgaaagccgttatgcaaaaaaagatatggaccgcgaacgtgtgaaagtccgtatgatgctcgagcagcaaaaatttcatagcgacatccaggtaaatctcgattctagcgctgcagggaccgaaactggaggccaagttgtttcagaatctcagaaatttaccgaacgtaatcgtcagattaaacagtga

(19) United States
(12) Patent Application Publication
(10) Pub. No.: US 2022/0088092 A1
(43) Pub. Date: Mar. 24, 2022

(54) COMPOSITIONS AND METHODS FOR INCREASING THE VIABILITY OF FREEZE-DRIED PROBIOTICS

(71) Applicants: THE BROAD INSTITUTE, INC., Cambridge, MA (US); MASSACHUSETTS INSTITUTE OF TECHNOLOGY, Cambridge, MA (US)

(72) Inventors: Daniel S. Collins, Cambridge, MA (US); Jonathan M. Stokes, Cambridge, MA (US); Luis R. Soenksen, Cambridge, MA (US); James J. Collins, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 17/424,291

(22) PCT Filed: Jan. 21, 2020
(86) PCT No.: PCT/US2020/014317

§ 371 Date: Jul. 20, 2021

Related U.S. Application Data
(60) Provisional application No. 62/794,916, filed on Jan. 21, 2019.

(51) Int. Cl. A61K 35/747; A61K 9/00; A61K 35/745; A61K 35/744; A61K 35/741; A61K 36/064; A61K 47/42; A61K 9/19

(52) U.S. Cl. CPC A61K 35/747; A61K 9/0053; A61K 35/745; A61K 9/19; A61K 35/741; A61K 36/064; A61K 47/42; A61K 35/744

(57) ABSTRACT

The present disclosure relates to compositions, methods, and kits for increasing the viability of bacteria that have been subjected to freeze-drying/lyophilization. In particular, the disclosure relates to compositions and methods for increasing the viability of living medicines (e.g., probiotics) during lyophilization, oral rehydration, and passage through the gastrointestinal tract

### CROSS REFERENCE TO RELATED APPLICATION

This application claims the benefit of U.S. Provisional Application No. 62/794,916, filed Jan. 21, 2019, entitled “Compositions and Methods for Increasing the Viability of Freeze-Dried Probiotics,” the entire contents of which are incorporated herein by reference.

### STATEMENT REGARDING FEDERALLY SPONSORED RESEARCH

This invention was made with government support under Grant No. HDTRA1-1-14-0006 awarded by the Department of Defense. The government has certain rights in the invention.

### FIELD OF THE DISCLOSURE

The disclosure relates generally to compositions, methods, and kits for increasing the viability of bacteria that have been subjected to freeze-drying/lyophilization. More particularly, the disclosure relates to compositions and methods for increasing the viability of living medicines (e.g., probiotics) during lyophilization, oral rehydration, and passage through the gastrointestinal tract.

### BACKGROUND OF THE DISCLOSURE

Probiotics are live microorganisms (e.g., living medicines) that provide health benefits in both animals and humans when consumed. Exemplary strains of probiotic bacteria may be derived from a number of bacterial genera including, for example but not limited to, Bacillus, Bifidobacterium, Enterococcus, Escherichia, Lactobacillus, Propionibacterium, and Saccharomyces (a yeast), with Lactobacillus and Bifidobacterium being the most common. The ability of probiotics to provide these health benefits is believed to result from their ability to improve or restore gut flora (e.g. probiotic bacteria). Probiotic bacteria are believed to exert a number of beneficial effects, including: competitive exclusion of infective bacterial species, reduction of putrefactive and genotoxic intestinal reactions, and generation of antimicrobial agents such as, for example, bacteriocins.

A typical dietary dose of probiotic bacteria may be about 10 million bacteria per gram of food, which is not difficult to produce from a technical standpoint. Natural and engineered probiotics are increasingly being used as living medicines for a variety of clinical applications. Depending on the strain and the number of organisms present in a specific probiotic, different pharmaceutical formulations may be used to maximize the yield and efficacy of such products. Currently, probiotics are available in various formats, including liquid tablets, supplemented dairy products, and freeze-dried pills. Therapeutic probiotics are commonly administered in liquid form, which require refrigeration for distribution and storage, and lead to tolerability issues for patients. Freeze-dried probiotics are among the most convenient and desirable presentations of these therapeutics. Unfortunately, significant decreases in probiotic bacterial population size and/or viability may occur during manufacturing, freeze-drying/lyophilization, and storing the probiotic bacteria. A need exists for agents and methods to increase/enhance the viability of freeze-dried/lyophilized probiotics.

### BRIEF SUMMARY OF THE DISCLOSURE

The present disclosure is based, at least in part, on the discovery that recombinant bacteria (e.g., probiotic bacteria) expressing one or more tardigrade-specific intrinsically disordered proteins (TSIDPs) such as, for example, TSIDPs 77580, 89226, 94063, 106094, and/or 107838 impart desiccation resistant characteristics onto the recombinant bacteria that greatly enhance viability after freeze-drying/lyophilization. In particular, the disclosure provides for the expression and/or addition of one or more TSIDPs (e.g., naturally occurring or engineered) within and/or around natural, engineered, or synthetic probiotic organisms, in order to enhance viability during lyophilization, oral rehydration, and passage through the gastrointestinal tract. Additionally, the present disclosure provides recombinant probiotic bacteria that may be used to enhance access, ease of use, and efficacy of probiotics used for the treatment of rare genetic diseases like urea cycle disorders (UCD) and phenylketonuria (PKU), as well as inflammatory bowel disease (IBD) and certain cancers.

In one aspect, the instant disclosure provides a composition that includes a probiotic bacteria population and one or more intrinsically disordered proteins (IDPs) possessing cryoprotective and/or vitrification properties.

In a related aspect, the instant disclosure provides a composition that includes a probiotic bacteria population and one or more tardigrade-specific intrinsically disordered proteins (TSIDPs).

In one embodiment, bacteria of the probiotic bacteria population express the one or more TSIDPs.

In certain embodiments, the probiotic bacteria population includes one or more bacterial species of the following genera: Lactobacillus, Bifidobacterium, Enterococcus, Streptococcus, Pediococcus, Leuconostoc, Bacillus, Escherichia, and/or Saccharomyces. Optionally, the one or more probiotic bacterial species include a Lactobacillus and/or a Bifidobacterium species.

In some embodiments, the TSIDPs are one or more of: tardigrade protein 77580, tardigrade protein 89226, tardigrade protein 94063, tardigrade protein 106094 and/or tardigrade protein 107838, including any and all combinations thereof.

In one embodiment, the probiotic bacteria population and the one or more TSIDPs are present in an admixture.

Another aspect of the disclosure provides a preparation for oral delivery to a subject, where the preparation includes one or more recombinant probiotic bacteria that express one or more tardigrade-specific intrinsically disordered proteins (TSIDPs).

In one embodiment, the one or more recombinant probiotic bacteria express the one or more TSIDPs.

An additional aspect of the instant disclosure provides a preparation for oral delivery that includes freeze-dried recombinant probiotic bacteria.

In one embodiment, the recombinant probiotic bacteria express one or more TSIDPs. Optionally, the TSIDPs are one or more of: tardigrade protein 77580, tardigrade protein 89226, tardigrade protein 94063, tardigrade protein 106094 and/or tardigrade protein 107838, and any and all combinations thereof.

Another aspect of the instant disclosure provides a freeze-dried preparation for oral delivery that includes one or more isolated tardigrade-specific intrinsically disordered protein (TSIDPs) and one or more isolated populations of probiotic bacteria.

A further aspect of the disclosure provides a pharmaceutical composition that includes an isolated tardigrade-specific intrinsically disordered protein (TSIDP), an isolated population of probiotic bacteria, and a pharmaceutically acceptable carrier.

An additional aspect of the disclosure provides a pharmaceutical composition that includes recombinant probiotic bacteria that express a tardigrade-specific intrinsically disordered protein (TSIDP), and a pharmaceutically acceptable carrier.

Another aspect of the instant disclosure provides a composition that includes a probiotic bacteria population and one or more natural or synthetic intrinsically disordered proteins (IDPs) that possesses cryoprotective and/or vitrification properties.

In certain embodiments, bacteria of the probiotic bacteria population express the one or more natural or synthetic IDPs. Optionally, the one or more natural or synthetic IDPs are prokaryotic or eukaryotic proteins that possess cryoprotective and/or vitrification properties.

In one embodiment, the probiotic bacteria population and the one or more IDPs are present in an admixture.

An additional aspect of the disclosure provides a preparation for oral delivery to a subject, the preparation including one or more recombinant probiotic bacteria that express one or more intrinsically disordered proteins (IDPs).

In certain embodiments, the one or more IDPs possess cryoprotective and/or vitrification properties.

In one embodiment, the one or more IDPs are prokaryotic or eukaryotic proteins that possess cryoprotective or vitrification properties, and/or a combination thereof.

A further aspect of the instant disclosure provides a preparation for oral delivery that includes freeze-dried recombinant probiotic bacteria.

In one embodiment, the recombinant probiotic bacteria express one or more IDPs. Optionally, the one or more IDPs are prokaryotic or eukaryotic proteins that possess cryoprotective and/or vitrification properties, optionally including combinations thereof.

Another aspect of the disclosure provides a freeze-dried preparation for oral delivery that includes one or more isolated intrinsically disordered protein (IDPs) and one or more isolated populations of probiotic bacteria.

An additional aspect of the disclosure provides a pharmaceutical composition that includes an isolated intrinsically disordered protein (IDP), an isolated population of probiotic bacteria, and a pharmaceutically acceptable carrier.

A further aspect of the disclosure provides a pharmaceutical composition that includes recombinant probiotic bacteria that express an intrinsically disordered protein (IDP), and a pharmaceutically acceptable carrier.

Another aspect of the instant disclosure provides a method for treating or preventing a probiotic-treatable disease or disorder in a subject having or at risk of developing the probiotic-treatable disease or disorder, the method involving administering to the subject: (a) a pharmaceutical composition that includes an isolated IDP, an isolated population of probiotic bacteria, and a pharmaceutically acceptable carrier, or (b) a pharmaceutical composition that includes recombinant probiotic bacteria that express an IDP, and a pharmaceutically acceptable carrier.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. In some embodiments, administration is oral. Additionally, or alternatively, in some embodiments, administration is parenteral. In some embodiments, administration is intravenous.

By “CAHS 89226 nucleic acid molecule” is meant a polynucleotide encoding a tardigrade 89226 polypeptide. An exemplary CAHS 89226 nucleic acid molecule is:

>CAHS_89226
(SEQ ID NO: 1)
cgatcccgcgaaattaatacgactcactataggggaattgtgagcggata
acaattcccctctagaaataattttgtttaactttaagaaggagatatac
catgggcagcagcATGGCTACCAAGGAATCAAAATATGAACGGGTGGAGA
AAGTGAACGTTGATGCTGACGGGGCTACACTGGTCAAGAACATCGGCGAG
GATCGGGGGAAAGAAGACCCTGGCATGAACTTTCAAGACAAGCGCCCTGC
AAATCTTGTCCCAGGCGCCCCGGCCGGTGTTATCCCGAACCGCATCGAAT
CGCTGCCGACTGATCGCGCGGGCCAACGTCTGCGTGAACACCTGAGTGAG
TCGGAACGGCTCCGTGTGTCGCGTTCGTCCACCAGTAGTAAATCGAGTTC
GTTCGTCGAACCCTCCCTGAAATATCGCGGGGAAATCGGACCGATCGGTA
AAAACGGTGAGTTCGTGGCGAGCAGCAATCGGCAAAATAGTAGTTCCAAC
GTCAGCAGTAGTGATAACAGCGAGCGCGCCAGCCCAGCCTCTCGCAATTC
GAATCCAGGCATGAACAACGGAATGACAACACAACGCACTACCGTGATCA
CCGAATCGAGCGTGCAGGGCCTGGGTGCGCAGCGGACCGTTCCGATTCAG
CCGCATCAGCAGCGCGAAGATCATGAAGTGATTACGCATGAATCTCATGC
TCGCGCTCCCGAGACTACGGTGGTGACAATCCCGACAACGCGCTTTGAAA
GCGCGCAGCTGGAATCTCGCCGCGACGGCCGCACCTATACCGAGGATAAA
GAACTCACCATTCCGGCGCCGGTGGTGGCGCCGCAGATTCATGCGCACCA
GCAAGTGAATATGTCGGGCGGGACGTCGGCTACAATTCACGCCACTACGG
ATCTTCATTTAGCGTCAGAGGCGCAGATCAATGACATGGGCCCGGAGGAG
TATGAGCGCTATCGGGCGAAAGTAGAGGCGCTGGCTCGCATTCATGAAGA
TGAAACAAGCCGTAAGGCGGCAGCATATCGTAACGCCGTCGAGGCAGACG
CAGAATTGATTCGCCAGACTCTGGAACGGCAGCATATGCGGGATATCGAG
TTCCGCAAGGATCTGGTCGAGAGTTCCGTTGATCGTCAACAGCAAGAGAT
CCGCTTAGAAGCCGAGTATGCGATGCGTGCCCTGGAGCAGGAGCGTGTTA
ATGCGCGCGCGGCGCTCGATCAGGCGATGGCGAGTACCAACATTGATGTC
AACATTGATTCGGCAATCGGCACGACGCACTCGCAGGGACGCGTTACAAC
CACCTCCGAGTCACGGACATCACAAGCCCGCGGTCCAGCAACCGCAGCAG
TCATCTAAgagatccggctgctaacaaagcccgaaaggaagctgagttgg
ctgctgccaccgctgagcaataactagcataaccccttggggcctctaaa
cgggtcttgaggggttttttgctgaaaggaggaactatatccggattggc
gaatgggacgcgccctgtagcggcgcattaagcgcggcgggtgtggtggt
tacgcgcagcgtgaccgctacacttgccagcgccctagcgcccgctcctt
tcgctttcttcccttcctttctcgccacgttcgccggctttccccgtcaa
gctctaaatcgggggctccctttagggttccgatttagtgctttacggca
cctcgaccccaaaaaacttgattagggtgatggttcacgtagtgggccat
cgccctgatagacggtttttcgccctttgacgttggagtccacgttcttt
aatagtggactcttgttccaaactggaacaacactcaaccctatctcggt
ctattcttttgatttataagggattttgccgatttcggcctattggttaa
aaaatgagctgatttaacaaaaatttaacgcgaattttaacaaaatatta
acgtttacaatttcaggtggcacttttcggggaaatgtgcgcggaacccc
tatttgtttatttttctaaatacattcaaatatgtatccgctcatgaatt
aattcttagaaaaactcatcgagcatcaaatgaaactgcaatttattcat
atcaggattatcaataccatatttttgaaaaagccgtttctgtaatgaag
gagaaaactcaccgaggcagttccataggatggcaagatcctggtatcgg
tctgcgattccgactcgtccaacatcaatacaacctattaatttcccctc
gtcaaaaataaggttatcaagtgagaaatcaccatgagtgacgactgaat
ccggtgagaatggcaaaagtttatgcatttctttccagacttgttcaaca
ggccagccattacgctcgtcatcaaaatcactcgcatcaaccaaaccgtt
attcattcgtgattgcgcctgagcgagacgaaatacgcgatcgctgttaa
aaggacaattacaaacaggaatcgaatgcaaccggcgcaggaacactgcc
agcgcatcaacaatattttcacctgaatcaggatattcttctaatacctg
gaatgctgttttcccggggatcgcagtggtgagtaaccatgcatcatcag
gagtacggataaaatgcttgatggtcggaagaggcataaattccgtcagc
cagtttagtctgaccatctcatctgtaacatcattggcaacgctaccttt
gccatgtttcagaaacaactctggcgcatcgggcttcccatacaatcgat
agattgtcgcacctgattgcccgacattatcgcgagcccatttataccca
tataaatcagcatccatgttggaatttaatcgcggcctagagcaagacgt
ttcccgttgaatatggctcataacaccccttgtattactgtttatgtaag
cagacagttttattgttcatgaccaaaatcccttaacgtgagttttcgtt
ccactgagcgtcagaccccgtagaaaagatcaaaggatcttcttgagatc
ctttttttctgcgcgtaatctgctgcttgcaaacaaaaaaaccaccgcta
ccagcggtggtttgtttgccggatcaagagctaccaactctttttccgaa
ggtaactggcttcagcagagcgcagataccaaatactgtccttctagtgt
agccgtagttaggccaccacttcaagaactctgtagcaccgcctacatac
ctcgctctgctaatcctgttaccagtggctgctgccagtggcgataagtc
gtgtcttaccgggttggactcaagacgatagttaccggataaggcgcagc
ggtcgggctgaacggggggttcgtgcacacagcccagcttggagcgaacg
acctacaccgaactgagatacctacagcgtgagctatgagaaagcgccac
gcttcccgaagggagaaaggcggacaggtatccggtaagcggcagggtcg
gaacaggagagcgcacgagggagcttccagggggaaacgcctggtatctt
tatagtcctgtcgggtttcgccacctctgacttgagcgtcgatttttgtg
atgctcgtcaggggggcggagcctatggaaaaacgccagcaacgcggcct
ttttacggttcctggccttttgctggccttttgctcacatgttctttcct
gcgttatcccctgattctgtggataaccgtattaccgcctttgagtgagc
tgataccgctcgccgcagccgaacgaccgagcgcagcgagtcagtgagcg
aggaagcggaagagcgcctgatgcggtattttctccttacgcatctgtgc
ggtatttcacaccgcatatatggtgcactctcagtacaatctgctctgat
gccgcatagttaagccagtatacactccgctatcgctacgtgactgggtc
atggctgcgccccgacacccgccaacacccgctgacgcgccctgacgggc
ttgtctgctcccggcatccgcttacagacaagctgtgaccgtctccggga
gctgcatgtgtcagaggttttcaccgtcatcaccgaaacgcgcgaggcag
ctgcggtaaagctcatcagcgtggtcgtgaagcgattcacagatgtctgc
ctgttcatccgcgtccagctcgttgagtttctccagaagcgttaatgtct
ggcttctgataaagcgggccatgttaagggcggttttttcctgtttggtc
actgatgcctccgtgtaagggggatttctgttcatgggggtaatgatacc
gatgaaacgagagaggatgctcacgatacgggttactgatgatgaacatg
cccggttactggaacgttgtgagggtaaacaactggcggtatggatgcgg
cgggaccagagaaaaatcactcagggtcaatgccagcgcttcgttaatac
agatgtaggtgttccacagggtagccagcagcatcctgcgatgcagatcc
ggaacataatggtgcagggcgctgacttccgcgtttccagactttacgaa
acacggaaaccgaagaccattcatgttgttgctcaggtcgcagacgtttt
gcagcagcagtcgcttcacgttcgctcgcgtatcggtgattcattctgct
aaccagtaaggcaaccccgccagcctagccgggtcctcaacgacaggagc
acgatcatgcgcacccgtggggccgccatgccggcgataatggcctgctt
ctcgccgaaacgtttggtggcgggaccagtgacgaaggcttgagcgaggg
cgtgcaagattccgaataccgcaagcgacaggccgatcatcgtcgcgctc
cagcgaaagcggtcctcgccgaaaatgacccagagcgctgccggcacctg
tcctacgagttgcatgataaagaagacagtcataagtgcggcgacgatag
tcatgccccgcgcccaccggaaggagctgactgggttgaaggctctcaag
ggcatcggtcgagatcccggtgcctaatgagtgagctaacttacattaat
tgcgttgcgctcactgcccgctttccagtcgggaaacctgtcgtgccagc
tgcattaatgaatcggccaacgcgcggggagaggcggtttgcgtattggg
cgccagggtggtttttcttttcaccagtgagacgggcaacagctgattgc
ccttcaccgcctggccctgagagagttgcagcaagcggtccacgctggtt
tgccccagcaggcgaaaatcctgtttgatggtggttaacggcgggatata
acatgagctgtcttcggtatcgtcgtatcccactaccgagatatccgcac
caacgcgcagcccggactcggtaatggcgcgcattgcgcccagcgccatc
tgatcgttggcaaccagcatcgcagtgggaacgatgccctcattcagcat
ttgcatggtttgttgaaaaccggacatggcactccagtcgccttcccgtt
ccgctatcggctgaatttgattgcgagtgagatatttatgccagccagcc
agacgcagacgcgccgagacagaacttaatgggcccgctaacagcgcgat
ttgctggtgacccaatgcgaccagatgctccacgcccagtcgcgtaccgt
cttcatgggagaaaataatactgttgatgggtgtctggtcagagacatca
agaaataacgccggaacattagtgcaggcagcttccacagcaatggcatc
ctggtcatccagcggatagttaatgatcagcccactgacgcgttgcgcga
gaagattgtgcaccgccgctttacaggcttcgacgccgcttcgttctacc
atcgacaccaccacgctggcacccagttgatcggcgcgagatttaatcgc
cgcgacaatttgcgacggcgcgtgcagggccagactggaggtggcaacgc
caatcagcaacgactgtttgcccgccagttgttgtgccacgcggttggga
atgtaattcagctccgccatcgccgcttccactttttcccgcgttttcgc
agaaacgtggctggcctggttcaccacgcgggaaacggtctgataagaga
caccggcatactctgcgacatcgtataacgttactggtttcacattcacc
accctgaattgactctcttccgggcgctatcatgccataccgcgaaaggt
tttgcgccattcgatggtgtccgggatctcgacgctctcccttatgcgac
tcctgcattaggaagcagcccagtagtaggttgaggccgttgagcaccgc
cgccgcaaggaatggtgcatgcaaggagatggcgcccaacagtcccccgg
ccacggggcctgccaccatacccacgccgaaacaagcgctcatgagcccg
aagtggcgagcccgatcttccccatcggtgatgtcggcgatataggcgcc
agcaaccgcacctgtggcgccggtgatgccggccacgatgcgtccggcgt
agaggatcgagatct

By “CAHS 94063 nucleic acid molecule” is meant a polynucleotide encoding a tardigrade 94063 polypeptide. An exemplary CAHS 94063 nucleic acid molecule is:

>CAHS_94063
(SEQ ID NO: 2)
cgatcccgcgaaattaatacgactcactataggggaattgtgagcggata
acaattcccctctagaaataattttgtttaactttaagaaggagatatac
catgggcagcagcATGTCAGGGCGTAACGTGGAGTCCCATATGGAGCGCA
ACGAAAAAGTGGTAGTGAATAACTCCGGTCATGCGGACGTGAAAAAACAG
CAACAACAGGTTGAACATACGGAGTTCACGCATACAGAAGTAAAAGCCCC
GCTCATCCACCCAGCCCCTCCGATTATTTCGACTGGCGCCGCGGGCTTAG
CGGAGGAAATTGTGGGCCAGGGTTTTACTGCGTCAGCAGCGCGTATCTCA
GGTGGCACTGCCGAAGTGCATCTGCAGCCGTCAGCGGCGATGACAGAAGA
AGCTCGCCGTGATCAGGAACGTTATCGGCAGGAGCAGGAAAGTATTGCGA
AACAACAGGAACGGGAAATGGAAAAGAAGACCGAAGCGTACCGCAAGACG
GCGGAGGCGGAAGCTGAAAAAATTCGTAAAGAACTGGAAAAACAACATGC
GCGCGATGTCGAATTCCGTAAAGATCTGATCGAATCCACGATCGATCGTC
AGAAACGTGAAGTGGATCTGGAAGCGAAAATGGCTAAACGCGAGTTAGAT
CGTGAAGGTCAGCTGGCTAAAGAAGCCCTGGAACGCTCTCGGTTAGCCAC
GAACGTCGAAGTTAACTTCGATTCGGCAGCCGGGCATACAGTCAGTGGAG
GGACCACTGTTAGCACTAGCGATAAGATGGAAATTAAACGCAACTAAgag
atccggctgctaacaaagcccgaaaggaagctgagttggctgctgccacc
gctgagcaataactagcataaccccttggggcctctaaacgggtcttgag
gggttttttgctgaaaggaggaactatatccggattggcgaatgggacgc
gccctgtagcggcgcattaagcgcggcgggtgtggtggttacgcgcagcg
tgaccgctacacttgccagcgccctagcgcccgctcctttcgctttcttc
ccttcctttctcgccacgttcgccggctttccccgtcaagctctaaatcg
ggggctccctttagggttccgatttagtgctttacggcacctcgacccca
aaaaacttgattagggtgatggttcacgtagtgggccatcgccctgatag
acggtttttcgccctttgacgttggagtccacgttctttaatagtggact
cttgttccaaactggaacaacactcaaccctatctcggtctattcttttg
atttataagggattttgccgatttcggcctattggttaaaaaatgagctg
atttaacaaaaatttaacgcgaattttaacaaaatattaacgtttacaat
ttcaggtggcacttttcggggaaatgtgcgcggaacccctatttgtttat
ttttctaaatacattcaaatatgtatccgctcatgaattaattcttagaa
aaactcatcgagcatcaaatgaaactgcaatttattcatatcaggattat
caataccatatttttgaaaaagccgtttctgtaatgaaggagaaaactca
ccgaggcagttccataggatggcaagatcctggtatcggtctgcgattcc
gactcgtccaacatcaatacaacctattaatttcccctcgtcaaaaataa
ggttatcaagtgagaaatcaccatgagtgacgactgaatccggtgagaat
ggcaaaagtttatgcatttctttccagacttgttcaacaggccagccatt
acgctcgtcatcaaaatcactcgcatcaaccaaaccgttattcattcgtg
attgcgcctgagcgagacgaaatacgcgatcgctgttaaaaggacaatta
caaacaggaatcgaatgcaaccggcgcaggaacactgccagcgcatcaac
aatattttcacctgaatcaggatattcttctaatacctggaatgctgttt
tcccggggatcgcagtggtgagtaaccatgcatcatcaggagtacggata
aaatgcttgatggtcggaagaggcataaattccgtcagccagtttagtct
gaccatctcatctgtaacatcattggcaacgctacctttgccatgtttca
gaaacaactctggcgcatcgggcttcccatacaatcgatagattgtcgca
cctgattgcccgacattatcgcgagcccatttatacccatataaatcagc
atccatgttggaatttaatcgcggcctagagcaagacgtttcccgttgaa
tatggctcataacaccccttgtattactgtttatgtaagcagacagtttt
attgttcatgaccaaaatcccttaacgtgagttttcgttccactgagcgt
cagaccccgtagaaaagatcaaaggatcttcttgagatcctttttttctg
cgcgtaatctgctgcttgcaaacaaaaaaaccaccgctaccagcggtggt
ttgtttgccggatcaagagctaccaactctttttccgaaggtaactggct
tcagcagagcgcagataccaaatactgtccttctagtgtagccgtagtta
ggccaccacttcaagaactctgtagcaccgcctacatacctcgctctgct
aatcctgttaccagtggctgctgccagtggcgataagtcgtgtcttaccg
ggttggactcaagacgatagttaccggataaggcgcagcggtcgggctga
acggggggttcgtgcacacagcccagcttggagcgaacgacctacaccga
actgagatacctacagcgtgagctatgagaaagcgccacgcttcccgaag
ggagaaaggcggacaggtatccggtaagcggcagggtcggaacaggagag
cgcacgagggagcttccagggggaaacgcctggtatctttatagtcctgt
cgggtttcgccacctctgacttgagcgtcgatttttgtgatgctcgtcag
gggggcggagcctatggaaaaacgccagcaacgcggcctttttacggttc
ctggccttttgctggccttttgctcacatgttctttcctgcgttatcccc
tgattctgtggataaccgtattaccgcctttgagtgagctgataccgctc
gccgcagccgaacgaccgagcgcagcgagtcagtgagcgaggaagcggaa
gagcgcctgatgcggtattttctccttacgcatctgtgcggtatttcaca
ccgcatatatggtgcactctcagtacaatctgctctgatgccgcatagtt
aagccagtatacactccgctatcgctacgtgactgggtcatggctgcgcc
ccgacacccgccaacacccgctgacgcgccctgacgggcttgtctgctcc
cggcatccgcttacagacaagctgtgaccgtctccgggagctgcatgtgt
cagaggttttcaccgtcatcaccgaaacgcgcgaggcagctgcggtaaag
ctcatcagcgtggtcgtgaagcgattcacagatgtctgcctgttcatccg
cgtccagctcgttgagtttctccagaagcgttaatgtctggcttctgata
aagcgggccatgttaagggcggttttttcctgtttggtcactgatgcctc
cgtgtaagggggatttctgttcatgggggtaatgataccgatgaaacgag
agaggatgctcacgatacgggttactgatgatgaacatgcccggttactg
gaacgttgtgagggtaaacaactggcggtatggatgcggcgggaccagag
aaaaatcactcagggtcaatgccagcgcttcgttaatacagatgtaggtg
ttccacagggtagccagcagcatcctgcgatgcagatccggaacataatg
gtgcagggcgctgacttccgcgtttccagactttacgaaacacggaaacc
gaagaccattcatgttgttgctcaggtcgcagacgttttgcagcagcagt
cgcttcacgttcgctcgcgtatcggtgattcattctgctaaccagtaagg
caaccccgccagcctagccgggtcctcaacgacaggagcacgatcatgcg
cacccgtggggccgccatgccggcgataatggcctgcttctcgccgaaac
gtttggtggcgggaccagtgacgaaggcttgagcgagggcgtgcaagatt
ccgaataccgcaagcgacaggccgatcatcgtcgcgctccagcgaaagcg
gtcctcgccgaaaatgacccagagcgctgccggcacctgtcctacgagtt
gcatgataaagaagacagtcataagtgcggcgacgatagtcatgccccgc
gcccaccggaaggagctgactgggttgaaggctctcaagggcatcggtcg
agatcccggtgcctaatgagtgagctaacttacattaattgcgttgcgct
cactgcccgctttccagtcgggaaacctgtcgtgccagctgcattaatga
atcggccaacgcgcggggagaggcggtttgcgtattgggcgccagggtgg
tttttcttttcaccagtgagacgggcaacagctgattgcccttcaccgcc
tggccctgagagagttgcagcaagcggtccacgctggtttgccccagcag
gcgaaaatcctgtttgatggtggttaacggcgggatataacatgagctgt
cttcggtatcgtcgtatcccactaccgagatatccgcaccaacgcgcagc
ccggactcggtaatggcgcgcattgcgcccagcgccatctgatcgttggc
aaccagcatcgcagtgggaacgatgccctcattcagcatttgcatggttt
gttgaaaaccggacatggcactccagtcgccttcccgttccgctatcggc
tgaatttgattgcgagtgagatatttatgccagccagccagacgcagacg
cgccgagacagaacttaatgggcccgctaacagcgcgatttgctggtgac
ccaatgcgaccagatgctccacgcccagtcgcgtaccgtcttcatgggag
aaaataatactgttgatgggtgtctggtcagagacatcaagaaataacgc
cggaacattagtgcaggcagcttccacagcaatggcatcctggtcatcca
gcggatagttaatgatcagcccactgacgcgttgcgcgagaagattgtgc
accgccgctttacaggcttcgacgccgcttcgttctaccatcgacaccac
cacgctggcacccagttgatcggcgcgagatttaatcgccgcgacaattt
gcgacggcgcgtgcagggccagactggaggtggcaacgccaatcagcaac
gactgtttgcccgccagttgttgtgccacgcggttgggaatgtaattcag
ctccgccatcgccgcttccactttttcccgcgttttcgcagaaacgtggc
tggcctggttcaccacgcgggaaacggtctgataagagacaccggcatac
tctgcgacatcgtataacgttactggtttcacattcaccaccctgaattg
actctcttccgggcgctatcatgccataccgcgaaaggttttgcgccatt
cgatggtgtccgggatctcgacgctctcccttatgcgactcctgcattag
gaagcagcccagtagtaggttgaggccgttgagcaccgccgccgcaagga
atggtgcatgcaaggagatggcgcccaacagtcccccggccacggggcct
gccaccatacccacgccgaaacaagcgctcatgagcccgaagtggcgagc
ccgatcttccccatcggtgatgtcggcgatataggcgccagcaaccgcac
ctgtggcgccggtgatgccggccacgatgcgtccggcgtagaggatcgag
atct

By “CAHS 106094 nucleic acid molecule” is meant a polynucleotide encoding a tardigrade 106094 polypeptide. An exemplary CAHS 106094 nucleic acid molecule is:

>CAHS_106094
(SEQ ID NO: 3)
cgatcccgcgaaattaatacgactcactataggggaattgtgagcggata
acaattcccctctagaaataattttgtttaactttaagaaggagatatac
catgggcagcagcATGGAGGCCATGAATATGAATATCCCCCGCGATGCCA
TGTTTGTTCCGCCACCGGAATCTGAGCAAAATGGGTATCATGAGAAGTCA
GAAGTTCAGCAAACAAGTTATATGCAGAGTCAAGTCAAAGTGCCACATTA
TAATTTCCCGACACCATATTTTACGACTTCCTTTTCTGCGCAAGAGCTGC
TGGGCGAAGGGTTTCAAGCCTCAATTTCCCGTATTTCAGCCGTTACGGAA
GACATGCAGAGCATGGAAATCCCGGAGTTCGTTGAAGAGGCCCGCCGTGA
TTACGCAGCCAAAACACGTGAAAATGAGATGCTGGGGCAACAATATGAAA
AAGAGCTGGAACGTAAGTCCGAAGCCTACCGCAAACATCAGGAAGTAGAG
GCCGACAAAATCCGCAAAGAACTTGAAAAACAGCATATGCGTGATATTGA
ATTTCGGAAAGAAATTGCAGAACTGGCGATTGAGAACCAAAAACGTATGA
TCGATCTTGAATGCCGCTATGCAAAAAAAGACATGGACCGGGAACGCACA
AAAGTTCGTATGATGCTGGAGCAACAGAAATTCCATAGTGATATCCAGGT
AAATCTGGATTCTTCTGCGGCTGGGACCGAGAGCGGAGGTCATGTAGTGA
GCCAGTCTGAAAAGTTCACCGAACGTAACCGCGAGATGAAACGCTAAgag
atccggctgctaacaaagcccgaaaggaagctgagttggctgctgccacc
gctgagcaataactagcataaccccttggggcctctaaacgggtcttgag
gggttttttgctgaaaggaggaactatatccggattggcgaatgggacgc
gccctgtagcggcgcattaagcgcggcgggtgtggtggttacgcgcagcg
tgaccgctacacttgccagcgccctagcgcccgctcctttcgctttcttc
ccttcctttctcgccacgttcgccggctttccccgtcaagctctaaatcg
ggggctccctttagggttccgatttagtgctttacggcacctcgacccca
aaaaacttgattagggtgatggttcacgtagtgggccatcgccctgatag
acggtttttcgccctttgacgttggagtccacgttctttaatagtggact
cttgttccaaactggaacaacactcaaccctatctcggtctattcttttg
atttataagggattttgccgatttcggcctattggttaaaaaatgagctg
atttaacaaaaatttaacgcgaattttaacaaaatattaacgtttacaat
ttcaggtggcacttttcggggaaatgtgcgcggaacccctatttgtttat
ttttctaaatacattcaaatatgtatccgctcatgaattaattcttagaa
aaactcatcgagcatcaaatgaaactgcaatttattcatatcaggattat
caataccatatttttgaaaaagccgtttctgtaatgaaggagaaaactca
ccgaggcagttccataggatggcaagatcctggtatcggtctgcgattcc
gactcgtccaacatcaatacaacctattaatttcccctcgtcaaaaataa
ggttatcaagtgagaaatcaccatgagtgacgactgaatccggtgagaat
ggcaaaagtttatgcatttctttccagacttgttcaacaggccagccatt
acgctcgtcatcaaaatcactcgcatcaaccaaaccgttattcattcgtg
attgcgcctgagcgagacgaaatacgcgatcgctgttaaaaggacaatta
caaacaggaatcgaatgcaaccggcgcaggaacactgccagcgcatcaac
aatattttcacctgaatcaggatattcttctaatacctggaatgctgttt
tcccggggatcgcagtggtgagtaaccatgcatcatcaggagtacggata
aaatgcttgatggtcggaagaggcataaattccgtcagccagtttagtct
gaccatctcatctgtaacatcattggcaacgctacctttgccatgtttca
gaaacaactctggcgcatcgggcttcccatacaatcgatagattgtcgca
cctgattgcccgacattatcgcgagcccatttatacccatataaatcagc
atccatgttggaatttaatcgcggcctagagcaagacgtttcccgttgaa
tatggctcataacaccccttgtattactgtttatgtaagcagacagtttt
attgttcatgaccaaaatcccttaacgtgagttttcgttccactgagcgt
cagaccccgtagaaaagatcaaaggatcttcttgagatcctttttttctg
cgcgtaatctgctgcttgcaaacaaaaaaaccaccgctaccagcggtggt
ttgtttgccggatcaagagctaccaactctttttccgaaggtaactggct
tcagcagagcgcagataccaaatactgtccttctagtgtagccgtagtta
ggccaccacttcaagaactctgtagcaccgcctacatacctcgctctgct
aatcctgttaccagtggctgctgccagtggcgataagtcgtgtcttaccg
ggttggactcaagacgatagttaccggataaggcgcagcggtcgggctga
acggggggttcgtgcacacagcccagcttggagcgaacgacctacaccga
actgagatacctacagcgtgagctatgagaaagcgccacgcttcccgaag
ggagaaaggcggacaggtatccggtaagcggcagggtcggaacaggagag
cgcacgagggagcttccagggggaaacgcctggtatctttatagtcctgt
cgggtttcgccacctctgacttgagcgtcgatttttgtgatgctcgtcag
gggggcggagcctatggaaaaacgccagcaacgcggcctttttacggttc
ctggccttttgctggccttttgctcacatgttctttcctgcgttatcccc
tgattctgtggataaccgtattaccgcctttgagtgagctgataccgctc
gccgcagccgaacgaccgagcgcagcgagtcagtgagcgaggaagcggaa
gagcgcctgatgcggtattttctccttacgcatctgtgcggtatttcaca
ccgcatatatggtgcactctcagtacaatctgctctgatgccgcatagtt
aagccagtatacactccgctatcgctacgtgactgggtcatggctgcgcc
ccgacacccgccaacacccgctgacgcgccctgacgggcttgtctgctcc
cggcatccgcttacagacaagctgtgaccgtctccgggagctgcatgtgt
cagaggttttcaccgtcatcaccgaaacgcgcgaggcagctgcggtaaag
ctcatcagcgtggtcgtgaagcgattcacagatgtctgcctgttcatccg
cgtccagctcgttgagtttctccagaagcgttaatgtctggcttctgata
aagcgggccatgttaagggcggttttttcctgtttggtcactgatgcctc
cgtgtaagggggatttctgttcatgggggtaatgataccgatgaaacgag
agaggatgctcacgatacgggttactgatgatgaacatgcccggttactg
gaacgttgtgagggtaaacaactggcggtatggatgcggcgggaccagag
aaaaatcactcagggtcaatgccagcgcttcgttaatacagatgtaggtg
ttccacagggtagccagcagcatcctgcgatgcagatccggaacataatg
gtgcagggcgctgacttccgcgtttccagactttacgaaacacggaaacc
gaagaccattcatgttgttgctcaggtcgcagacgttttgcagcagcagt
cgcttcacgttcgctcgcgtatcggtgattcattctgctaaccagtaagg
caaccccgccagcctagccgggtcctcaacgacaggagcacgatcatgcg
cacccgtggggccgccatgccggcgataatggcctgcttctcgccgaaac
gtttggtggcgggaccagtgacgaaggcttgagcgagggcgtgcaagatt
ccgaataccgcaagcgacaggccgatcatcgtcgcgctccagcgaaagcg
gtcctcgccgaaaatgacccagagcgctgccggcacctgtcctacgagtt
gcatgataaagaagacagtcataagtgcggcgacgatagtcatgccccgc
gcccaccggaaggagctgactgggttgaaggctctcaagggcatcggtcg
agatcccggtgcctaatgagtgagctaacttacattaattgcgttgcgct
cactgcccgctttccagtcgggaaacctgtcgtgccagctgcattaatga
atcggccaacgcgcggggagaggcggtttgcgtattgggcgccagggtgg
tttttcttttcaccagtgagacgggcaacagctgattgcccttcaccgcc
tggccctgagagagttgcagcaagcggtccacgctggtttgccccagcag
gcgaaaatcctgtttgatggtggttaacggcgggatataacatgagctgt
cttcggtatcgtcgtatcccactaccgagatatccgcaccaacgcgcagc
ccggactcggtaatggcgcgcattgcgcccagcgccatctgatcgttggc
aaccagcatcgcagtgggaacgatgccctcattcagcatttgcatggttt
gttgaaaaccggacatggcactccagtcgccttcccgttccgctatcggc
tgaatttgattgcgagtgagatatttatgccagccagccagacgcagacg
cgccgagacagaacttaatgggcccgctaacagcgcgatttgctggtgac
ccaatgcgaccagatgctccacgcccagtcgcgtaccgtcttcatgggag
aaaataatactgttgatgggtgtctggtcagagacatcaagaaataacgc
cggaacattagtgcaggcagcttccacagcaatggcatcctggtcatcca
gcggatagttaatgatcagcccactgacgcgttgcgcgagaagattgtgc
accgccgctttacaggcttcgacgccgcttcgttctaccatcgacaccac
cacgctggcacccagttgatcggcgcgagatttaatcgccgcgacaattt
gcgacggcgcgtgcagggccagactggaggtggcaacgccaatcagcaac
gactgtttgcccgccagttgttgtgccacgcggttgggaatgtaattcag
ctccgccatcgccgcttccactttttcccgcgttttcgcagaaacgtggc
tggcctggttcaccacgcgggaaacggtctgataagagacaccggcatac
tctgcgacatcgtataacgttactggtttcacattcaccaccctgaattg
actctcttccgggcgctatcatgccataccgcgaaaggttttgcgccatt
cgatggtgtccgggatctcgacgctctcccttatgcgactcctgcattag
gaagcagcccagtagtaggttgaggccgttgagcaccgccgccgcaagga
atggtgcatgcaaggagatggcgcccaacagtcccccggccacggggcct
gccaccatacccacgccgaaacaagcgctcatgagcccgaagtggcgagc
ccgatcttccccatcggtgatgtcggcgatataggcgccagcaaccgcac
ctgtggcgccggtgatgccggccacgatgcgtccggcgtagaggatcgag
atct

As used herein, the term “Intrinsically Disordered Protein” or “IDP” refers to a protein that lacks a fixed or ordered three-dimensional structure (Dunker et al. Journal of Molecular Graphics & Modelling. 19: 26-59; Dyson and Wright. Nature Reviews. Molecular Cell Biology. 6: 197-208; Dunker et al. Current Opinion in Structural Biology. 18: 756-64). IDPs cover a spectrum of states from fully unstructured to partially structured and include random coils, (pre-)molten globules, and large multi-domain proteins connected by flexible linkers. IDPs constitute one of the main types of protein (alongside globular, fibrous and membrane proteins; Andreeva et al. Nucleic Acids Research. 42: D310-4). In certain aspects of the instant disclosure, an IDP refers to a protein that lacks a fixed or ordered three-dimensional structure but that also possesses cryoprotective and/or vitrification properties (e.g., the ability to enhance the viability of associated biological compositions that are freeze-dried, lyophilized, etc.). One class of IDP is the class of tardigrade-specific intrinsically disordered proteins (TSIDPs), representative members of which have been identified herein to possess cryoprotective and/or vitrification properties.

By “control” or “reference” is meant a standard of comparison. In one aspect, as used herein, “changed as compared to a control” sample or subject is understood as having a level that is statistically different or biologically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

In some embodiments, a recombinant nucleic acid molecule of the invention can be an “expression cassette” or can be comprised within an expression cassette. As used herein, the term “expression cassette” means a recombinant nucleic acid molecule comprising a nucleotide sequence of interest (e.g., the TSIDPs nucleotide sequences disclosed herein and/or fragments thereof) that is operably associated with at least a control sequence (e.g., a promoter and/or enhancer). Thus, some embodiments of the invention provide expression cassettes designed to express the nucleotide sequences of the invention in a cell.

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression. An expression cassette also can optionally include a transcriptional and/or translational termination region (i.e., termination region) that is functional in the cell in which the nucleotide sequence of interest is to be expressed. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the host organism, or may be derived from another source (i.e., foreign or heterologous to the promoter, the nucleotide sequence of interest, the host organism, or any combination thereof). In addition, in some embodiments, a coding sequence's native transcription terminator can be used. An expression cassette of the invention also can include a nucleotide sequence for a selectable marker, which can be used to select a transformed organism and/or cell. As used herein, “selectable marker” means a nucleotide sequence that when expressed imparts a distinct phenotype to the transformed organism or cell expressing the marker and thus allows such transformed organisms or cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening. Of course, many examples of suitable selectable markers useful in various organisms are known in the art and can be used in the expression cassettes described herein.

In addition to expression cassettes, the nucleic acid molecules and nucleotide sequences described herein can be used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Vectors for use in transformation of animals, plants and other organisms are well known in the art. Non-limiting examples of general classes of vectors including but not limited to a viral vector, a plasmid vector, a phage vector, a phagemid vector, a cosmid vector, a fosmid vector, a bacteriophage, an artificial chromosome, or an Agrobacterium binary vector in double or single stranded linear or circular form which may or may not be self-transmissible or mobilizable. A vector as defined herein can transform prokaryotic or eukaryotic host either by integration into the cellular genome or exist extrachromosomally (e.g., an autonomous replicating plasmid with an origin of replication). Additionally, included are shuttle vectors by which is meant a DNA vehicle capable, naturally or by design, of replication in two different host organisms, which may be selected from prokaryotic and eukaryotic organisms. In some representative embodiments, the nucleic acid in the vector is under the control of, and operably linked to, an appropriate promoter or other regulatory elements for transcription in a host cell such as a microbial, e.g. bacterial, or an animal or a plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

The terms “isolated,” “purified”, or “biologically pure” refer to material that is free to varying degrees from components which normally accompany it as found in its native state. “Isolate” denotes a degree of separation from original source or surroundings. “Purify” denotes a degree of separation that is higher than isolation.

The term “probiotic” refers to bacterial genera that have a beneficial effect in animal organs, such as the human GI and vaginal tracts. The bacterial genera used most often as probiotics are lactobacilli and bifidobacteria; however, other beneficial bacterial species, such as S. thermophilis are also probiotics. After passage through the stomach and small intestine, some probiotics survive and become established transiently in the large bowel, where the colon's fermentation capacity is positively modified. See, e.g., Roberfroid, AM J CLIN NUTR 71 (SUPPL):1682S-1687S (2000). Exemplary probiotics therefore include any of a number of a phylogenetic spectrum of organisms including, but not limited to, species within the genera Lactobacillus, Bifidobacterium, Enterococcus, Streptococcus, Pediococcus, Leuconostoc, Bacillus, Escherichia, and Saccharomyces.

The probiotic species that may be used in the compositions of the present disclosure may be any known probiotic, such as, for example, L. acidophilus, L. bulgaricus, L. casei, L. paracasei, L. fermentum, L. plantarum, L. rhamnosus, L. salivarius, B. bifidum, B. infantis, B. animalis subsp. lactis, B. longum, S. thermophilis, E. faecalis, and E. faecium. It is to be understood that the foregoing list is intended only to be illustrative and not a limiting representation of the probiotics that may be included in the probiotic compositions of the present disclosure. In this respect, any additional probiotic species may also be used in the compositions of the present disclosure, such as, for example, any additional known and/or available lactobacillus or bifidobacterium species.

The term “probiotic-treatable disease or disorder” refers to any disease or disorder for which a positive outcome of probiotic administration might be expected. Exemplary types of “probiotic-treatable disease or disorder” include, but are not limited to, urea cycle disorder (UCD), phenylketonuria (PKU), inflammatory bowel disease (IBD) and certain cancers (e.g., cancers of the gastrointestinal tract, such as colorectal cancer, gastric cancer, esophageal cancer, etc.).

As used herein, the term “subject” includes humans and mammals (e.g., mice, rats, pigs, cats, dogs, nonhuman primates and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

Unless specifically stated or obvious from context, as used herein, the term “or” is understood to be inclusive. Unless specifically stated or obvious from context, as used herein, the terms “a,” “an,” and “the” are understood to be singular or plural.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable salts, esters, amides, and prodrugs” as used herein refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference.)

By “Tardigrade 77580 polypeptide” is meant a polypeptide or fragment thereof having at least about 85%, 86%, 87%, 88%, 89%, 90%, 91%, 92%, 93%, 94%, 95%, 96%, 97%, 98%, 99%, or 100% amino acid identity to NCBI Accession No. P0CU43 and having intrinsically disordered protein properties, the polypeptide having the structure:

>Tardigrade_77580
(SEQ ID NO: 4)
MSNYQQESSYQYSDRSNNGQQQEQQEKKEVEHSSYTHTDVKVNMPNLIAP
FISSSAGLAQELVGEGFQASVSRITGASGELTVIDTEAETEEARRDMEAK
AREQELLSRQFEKELERKTEAYRKQQEVETEKIRKELEKQHLRDVEFRKE
LMEQTIENQKRQIDLEARYAKKELERERNKVKRVLERSKFHTDIQVNMEA
AAGSTHSGSSSVAVSESEKFQTNN

By “Tardigrade 77580 nucleic acid molecule” is meant a polynucleotide encoding a Tardigrade 77580 polypeptide. An exemplary endogenous Tardigrade 77580 nucleic acid molecule is:

>endogenous_Tardigrade_77580_nucleic_acid
(SEQ ID NO: 5)
ATGTCTAACTACCAGCAAGAATCCAGCTATCAGTACAGCGACCGGAGCAA
CAATGGTCAACAGCAAGAGCAACAGGAGAAGAAGGAGGTCGAACACTCCA
GCTATACCCACACCGACGTCAAAGTGAACATGCCCAACTTAATCGCACCT
TTCATCAGCTCTTCCGCGGGTTTGGCTCAAGAACTGGTCGGGGAAGGTTT
CCAGGCGTCCGTCTCTCGCATCACCGGCGCATCCGGGGAGCTCACCGTCA
TCGACACCGAGGCCGAAACCGAGGAGGCACGTCGGGACATGGAAGCCAAG
GCCCGCGAGCAGGAGCTCCTGTCGCGACAGTTTGAGAAGGAGCTGGAGCG
AAAGACCGAAGCGTATCGCAAGCAGCAAGAAGTTGAGACCGAGAAGATCC
GGAAGGAACTTGAGAAGCAACATCTGCGGGATGTCGAGTTCCGCAAGGAG
TTGATGGAGCAGACCATCGAGAATCAGAAGCGTCAGATCGACCTGGAGGC
ACGCTATGCCAAGAAGGAGCTTGAGCGGGAACGGAACAAGGTCAAGCGTG
TGCTGGAACGCTCCAAATTCCACACCGATATCCAGGTCAACATGGAAGCC
GCTGCGGGTTCAACTCATTCCGGATCATCCAGCGTTGCCGTGTCGGAGTC
GGAAAAGTTCCAGACCAACAACTGA

By “Tardigrade 77580 nucleic acid molecule” is meant a polynucleotide encoding a Tardigrade 77580 polypeptide. An exemplary recombinant Tardigrade 77580 nucleic acid molecule is:

>recombinant_Tardigrade_77580_nucleic_acid
(SEQ ID NO: 6)
ATGAGTAACTATCAGCAAGAATCCAGCTATCAATATAGCGATCGCAGTAA
TAATGGCCAGCAACAAGAACAGCAAGAAAAGAAGGAAGTAGAACACTCGA
GTTACACGCACACTGACGTAAAAGTAAATATGCCGAACTTGATTGCCCCT
TTTATTTCATCTTCTGCGGGTCTGGCGCAGGAATTGGTCGGCGAGGGTTT
CCAAGCAAGCGTATCTCGTATCACTGGCGCATCGGGCGAATTAACTGTGA
TTGATACTGAAGCAGAAACCGAGGAAGCTCGCCGTGACATGGAAGCCAAG
GCACGTGAACAGGAGCTCCTGAGTCGTCAGTTTGAAAAAGAACTGGAACG
TAAAACCGAGGCCTATCGCAAACAGCAGGAAGTTGAAACTGAAAAAATCC
GCAAAGAATTAGAAAAACAGCATCTGCGCGATGTGGAGTTCCGTAAAGAA
CTCATGGAACAGACTATTGAAAACCAAAAACGCCAGATTGACCTCGAAGC
GCGCTACGCTAAGAAAGAGCTCGAGCGTGAGCGGAATAAAGTCAAACGCG
TTCTGGAACGTAGCAAATTTCATACAGACATCCAGGTAAACATGGAAGCG
GCTGCGGGTTCTACTCACAGCGGCTCAAGCTCTGTGGCTGTTAGCGAAAG
TGAAAAGTTCCAGACCAATAACTAA

By “Tardigrade 89226 polypeptide” is meant a polypeptide or fragment thereof having at least about 85%, 86%, 87%, 88%, 89%, 90%, 91%, 92%, 93%, 94%, 95%, 96%, 97%, 98%, 99%, or 100% amino acid identity to NCBI Accession No. POCU47 and having intrinsically disordered protein properties, the polypeptide having the structure:

>Tardigrade_89226
(SEQ ID NO: 7)
MATKESKYERVEKVNVDADGATLVKNIGEDRGKEDPGMNFQDKRPANLVP
GAPAGVIPNRIESLPTDRAGQRLREHLSESERLRVSRSSTSSKSSSFVEP
SLKYRGEIGPIGKNGEFVASSNRQNSSSNVSSSDNSERASPASRNSNPGM
NNGMTTQRTTVITESSVQGLGAQRTVPIQPHQQREDHEVITHESHARAPE
TTVVTIPTTRFESAQLESRRDGRTYTEDKELTIPAPVVAPQIHAHQQVNM
SGGTSATIHATTDLHLASEAQINDMGPEEYERYRAKVEALARIHEDETSR
KAAAYRNAVEADAELIRQTLERQHMRDIEFRKDLVESSVDRQQQEIRLEA
EYAMRALEQERVNARAALDQAMASTNIDVNIDSAIGTTHSQGRVTTTSES
RTSQARGPATAAVI

By “Tardigrade 89226 nucleic acid molecule” is meant a polynucleotide encoding a Tardigrade 89226 polypeptide. An exemplary endogenous Tardigrade 89226 nucleic acid molecule is:

>endogenous_Tardigrade_89226_nucleic_acid
(SEQ ID NO: 8)
ATGGCCACCAAGGAATCAAAGTATGAACGCGTGGAGAAGGTCAACGTGGA
CGCGGATGGCGCCACACTGGTCAAGAATATCGGCGAGGACCGCGGCAAGG
AGGATCCCGGGATGAATTTCCAGGACAAACGCCCGGCCAATTTGGTGCCC
GGAGCGCCGGCCGGAGTCATTCCCAACCGTATCGAGTCATTGCCGACGGA
TCGTGCCGGGCAACGTCTCCGTGAGCATCTCAGCGAGAGCGAACGCCTTC
GCGTCTCCCGCAGCAGCACCAGCAGCAAGTCTTCCAGCTTCGTGGAGCCC
AGCCTCAAGTACCGCGGTGAGATCGGACCGATCGGAAAAAACGGCGAGTT
CGTCGCCTCGTCGAACCGTCAGAACTCCAGCAGCAACGTCTCGTCCTCCG
ACAACAGCGAGCGTGCGTCTCCGGCGTCCCGCAACTCCAACCCCGGGATG
AACAACGGCATGACGACCCAACGCACCACGGTGATCACGGAGAGCTCGGT
TCAGGGCCTCGGCGCGCAACGCACCGTCCCGATCCAGCCGCATCAGCAGC
GCGAAGACCACGAGGTGATCACCCACGAATCGCATGCTCGAGCTCCGGAA
ACGACCGTGGTGACCATTCCCACGACGCGCTTCGAGTCCGCGCAGCTGGA
GTCGCGCCGCGACGGCAGAACCTACACCGAGGACAAGGAGCTGACCATTC
CGGCGCCGGTGGTCGCGCCGCAGATCCACGCCCACCAGCAGGTTAACATG
TCCGGAGGAACTTCGGCGACGATCCACGCCACGACCGATTTACATTTGGC
TAGCGAGGCACAGATCAATGACATGGGACCAGAGGAGTATGAGCGCTACC
GTGCCAAGGTGGAAGCCTTGGCTCGCATACATGAGGACGAAACGTCGCGC
AAAGCGGCGGCCTACCGTAATGCCGTTGAGGCCGATGCGGAACTCATCCG
CCAAACTCTGGAGCGCCAACACATGCGTGACATTGAATTCCGCAAGGATC
TAGTTGAAAGCTCGGTGGATCGTCAACAGCAGGAGATCCGCCTGGAGGCC
GAGTACGCCATGCGAGCCCTGGAACAGGAACGTGTCAATGCTCGCGCTGC
TCTTGACCAAGCCATGGCGTCGACGAACATCGACGTGAACATTGACTCCG
CTATCGGCACGACCCATTCCCAGGGAAGGGTTACGACGACCTCTGAAAGC
CGAACAAGTCAGGCACGTGGACCCGCCACTGCCGCAGTTATCTGA

By “Tardigrade 89226 nucleic acid molecule” is meant a polynucleotide encoding a Tardigrade 89226 polypeptide. An exemplary recombinant Tardigrade 89226 nucleic acid molecule is:

>recombinant_Tardigrade_89226_nucleic_acid
(SEQ ID NO: 9)
ATGGCTACCAAGGAATCAAAATATGAACGGGTGGAGAAAGTGAACGTTGA
TGCTGACGGGGCTACACTGGTCAAGAACATCGGCGAGGATCGGGGGAAAG
AAGACCCTGGCATGAACTTTCAAGACAAGCGCCCTGCAAATCTTGTCCCA
GGCGCCCCGGCCGGTGTTATCCCGAACCGCATCGAATCGCTGCCGACTGA
TCGCGCGGGCCAACGTCTGCGTGAACACCTGAGTGAGTCGGAACGGCTCC
GTGTGTCGCGTTCGTCCACCAGTAGTAAATCGAGTTCGTTCGTCGAACCC
TCCCTGAAATATCGCGGGGAAATCGGACCGATCGGTAAAAACGGTGAGTT
CGTGGCGAGCAGCAATCGGCAAAATAGTAGTTCCAACGTCAGCAGTAGTG
ATAACAGCGAGCGCGCCAGCCCAGCCTCTCGCAATTCGAATCCAGGCATG
AACAACGGAATGACAACACAACGCACTACCGTGATCACCGAATCGAGCGT
GCAGGGCCTGGGTGCGCAGCGGACCGTTCCGATTCAGCCGCATCAGCAGC
GCGAAGATCATGAAGTGATTACGCATGAATCTCATGCTCGCGCTCCCGAG
ACTACGGTGGTGACAATCCCGACAACGCGCTTTGAAAGCGCGCAGCTGGA
ATCTCGCCGCGACGGCCGCACCTATACCGAGGATAAAGAACTCACCATTC
CGGCGCCGGTGGTGGCGCCGCAGATTCATGCGCACCAGCAAGTGAATATG
TCGGGCGGGACGTCGGCTACAATTCACGCCACTACGGATCTTCATTTAGC
GTCAGAGGCGCAGATCAATGACATGGGCCCGGAGGAGTATGAGCGCTATC
GGGCGAAAGTAGAGGCGCTGGCTCGCATTCATGAAGATGAAACAAGCCGT
AAGGCGGCAGCATATCGTAACGCCGTCGAGGCAGACGCAGAATTGATTCG
CCAGACTCTGGAACGGCAGCATATGCGGGATATCGAGTTCCGCAAGGATC
TGGTCGAGAGTTCCGTTGATCGTCAACAGCAAGAGATCCGCTTAGAAGCC
GAGTATGCGATGCGTGCCCTGGAGCAGGAGCGTGTTAATGCGCGCGCGGC
GCTCGATCAGGCGATGGCGAGTACCAACATTGATGTCAACATTGATTCGG
CAATCGGCACGACGCACTCGCAGGGACGCGTTACAACCACCTCCGAGTCA
CGGACATCACAAGCCCGCGGTCCAGCAACCGCAGCAGTCATCTAA

By “Tardigrade 94063 polypeptide” is meant a polypeptide or fragment thereof having at least about 85%, 86%, 87%, 88%, 89%, 90%, 91%, 92%, 93%, 94%, 95%, 96%, 97%, 98%, 99%, or 100% amino acid identity to NCBI Accession No. POCU50 and having intrinsically disordered protein properties, the polypeptide having the structure:

>Tardigrade_94063
(SEQ ID NO: 10)
MSGRNVESHMERNEKVVVNNSGHADVKKQQQQVEHTEFTHTEVKAPLIHP
APPIISTGAAGLAEEIVGQGFTASAARISGGTAEVHLQPSAAMTEEARRD
QERYRQEQESIAKQQEREMEKKTEAYRKTAEAEAEKIRKELEKQHARDVE
FRKDLIESTIDRQKREVDLEAKMAKRELDREGQLAKEALERSRLATNVEV
NFDSAAGHTVSGGTTVSTSDKMEIKRN

By “Tardigrade 94063 nucleic acid molecule” is meant a polynucleotide encoding a Tardigrade 94063 polypeptide. An exemplary endogenous Tardigrade 94063 nucleic acid molecule is:

>endogenous_Tardigrade_94063_nucleic-acid
(SEQ ID NO: 11)
ATGTCTGGACGTAACGTAGAAAGCCACATGGAGCGGAATGAGAAGGTTGT
GGTCAACAATTCCGGTCATGCTGACGTGAAGAAGCAACAGCAACAAGTGG
AGCACACCGAATTCACCCACACCGAGGTTAAAGCCCCGTTGATCCATCCC
GCACCTCCGATCATCTCAACTGGAGCCGCCGGACTCGCCGAGGAGATTGT
GGGACAAGGGTTCACCGCGAGTGCCGCGCGCATCAGTGGAGGTACCGCTG
AAGTACATCTCCAGCCTTCGGCCGCAATGACGGAAGAGGCCCGTCGCGAT
CAAGAGCGCTACCGCCAGGAACAGGAGTCGATCGCCAAGCAGCAGGAACG
TGAGATGGAAAAGAAGACTGAGGCATACCGCAAGACCGCCGAGGCGGAAG
CTGAGAAGATCCGAAAGGAGCTGGAGAAGCAACACGCGCGTGATGTCGAG
TTCCGTAAGGATCTCATCGAGAGCACGATTGACCGGCAAAAACGCGAGGT
CGATCTGGAAGCGAAAATGGCCAAGCGGGAATTGGATCGTGAAGGGCAGT
TGGCCAAGGAGGCGTTGGAACGATCACGTTTGGCCACCAACGTTGAGGTC
AATTTCGACAGTGCAGCTGGTCATACCGTGTCCGGTGGGACGACCGTATC
CACTTCGGACAAGATGGAAATCAAGCGAAACTAG

By “Tardigrade 94063 nucleic acid molecule” is meant a polynucleotide encoding a Tardigrade 94063 polypeptide. An exemplary recombinant Tardigrade 94063 nucleic acid molecule is:

>recombinant_Tardigrade_94063_nucleic-acid
(SEQ ID NO: 12)
ATGTCAGGGCGTAACGTGGAGTCCCATATGGAGCGCAACGAAAAAGTGGT
AGTGAATAACTCCGGTCATGCGGACGTGAAAAAACAGCAACAACAGGTTG
AACATACGGAGTTCACGCATACAGAAGTAAAAGCCCCGCTCATCCACCCA
GCCCCTCCGATTATTTCGACTGGCGCCGCGGGCTTAGCGGAGGAAATTGT
GGGCCAGGGTTTTACTGCGTCAGCAGCGCGTATCTCAGGTGGCACTGCCG
AAGTGCATCTGCAGCCGTCAGCGGCGATGACAGAAGAAGCTCGCCGTGAT
CAGGAACGTTATCGGCAGGAGCAGGAAAGTATTGCGAAACAACAGGAACG
GGAAATGGAAAAGAAGACCGAAGCGTACCGCAAGACGGCGGAGGCGGAAG
CTGAAAAAATTCGTAAAGAACTGGAAAAACAACATGCGCGCGATGTCGAA
TTCCGTAAAGATCTGATCGAATCCACGATCGATCGTCAGAAACGTGAAGT
GGATCTGGAAGCGAAAATGGCTAAACGCGAGTTAGATCGTGAAGGTCAGC
TGGCTAAAGAAGCCCTGGAACGCTCTCGGTTAGCCACGAACGTCGAAGTT
AACTTCGATTCGGCAGCCGGGCATACAGTCAGTGGAGGGACCACTGTTAG
CACTAGCGATAAGATGGAAATTAAACGCAACTAA

By “Tardigrade 106094 polypeptide” is meant a polypeptide or fragment thereof having at least about 85%, 86%, 87%, 88%, 89%, 90%, 91%, 92%, 93%, 94%, 95%, 96%, 97%, 98%, 99%, or 100% amino acid identity to NCBI Accession No. POCU52 and having intrinsically disordered protein properties, the polypeptide having the structure:

>Tardigrade_106094
(SEQ ID NO: 13)
MEAMNMNIPRDAMFVPPPESEQNGYHEKSEVQQTSYMQSQVKVPHYNFPT
PYFTTSFSAQELLGEGFQASISRISAVTEDMQSMEIPEFVEEARRDYAAK
TRENEMLGQQYEKELERKSEAYRKHQEVEADKIRKELEKQHMRDIEFRKE
IAELAIENQKRMIDLECRYAKKDMDRERTKVRMMLEQQKFHSDIQVNLDS
SAAGTESGGHVVSQSEKFTERNREMKR

By “Tardigrade 106094 nucleic acid molecule” is meant a polynucleotide encoding a Tardigrade 106094 polypeptide. An exemplary endogenous Tardigrade 106094 nucleic acid molecule is:

>endogenous_Tardigrade_106094_nucleic_acid
(SEQ IS NO: 14)
ATGGAAGCGATGAATATGAACATTCCCCGGGATGCCATGTTTGTGCCGCC
GCCCGAGAGCGAGCAAAATGGGTACCATGAGAAGAGCGAGGTCCAGCAGA
CCAGCTACATGCAGTCGCAGGTTAAGGTGCCCCATTATAATTTCCCCACG
CCCTATTTCACCACATCTTTCTCGGCACAAGAGCTCCTTGGCGAGGGATT
TCAGGCATCGATTTCTCGCATCAGCGCCGTGACGGAAGATATGCAATCAA
TGGAGATCCCCGAGTTCGTCGAAGAAGCCCGTCGCGATTACGCCGCTAAG
ACCAGGGAGAATGAGATGCTGGGGCAGCAGTACGAGAAGGAGTTGGAGCG
GAAATCGGAGGCGTACCGAAAGCATCAGGAAGTGGAGGCCGACAAGATCC
GTAAAGAACTGGAGAAGCAGCACATGCGCGACATTGAGTTCCGCAAGGAG
ATCGCCGAGCTGGCCATCGAGAACCAGAAGCGCATGATCGACCTCGAATG
CCGCTATGCCAAAAAGGATATGGACCGTGAACGCACCAAGGTTCGAATGA
TGCTCGAACAACAAAAGTTCCACAGCGATATCCAGGTCAATCTGGATTCG
TCGGCTGCCGGCACGGAATCTGGTGGTCATGTGGTGTCACAGTCCGAGAA
GTTTACCGAACGAAACCGCGAGATGAAGCGATAA

By “Tardigrade 106094 nucleic acid molecule” is meant a polynucleotide encoding a Tardigrade 106094 polypeptide. An exemplary recombinant Tardigrade 106094 nucleic acid molecule is:

>recombinant_Tardigrade_106094_nucleic_acid
(SEQ ID NO: 15)
ATGGAGGCCATGAATATGAATATCCCCCGCGATGCCATGTTTGTTCCGCC
ACCGGAATCTGAGCAAAATGGGTATCATGAGAAGTCAGAAGTTCAGCAAA
CAAGTTATATGCAGAGTCAAGTCAAAGTGCCACATTATAATTTCCCGACA
CCATATTTTACGACTTCCTTTTCTGCGCAAGAGCTGCTGGGCGAAGGGTT
TCAAGCCTCAATTTCCCGTATTTCAGCCGTTACGGAAGACATGCAGAGCA
TGGAAATCCCGGAGTTCGTTGAAGAGGCCCGCCGTGATTACGCAGCCAAA
ACACGTGAAAATGAGATGCTGGGGCAACAATATGAAAAAGAGCTGGAACG
TAAGTCCGAAGCCTACCGCAAACATCAGGAAGTAGAGGCCGACAAAATCC
GCAAAGAACTTGAAAAACAGCATATGCGTGATATTGAATTTCGGAAAGAA
ATTGCAGAACTGGCGATTGAGAACCAAAAACGTATGATCGATCTTGAATG
CCGCTATGCAAAAAAAGACATGGACCGGGAACGCACAAAAGTTCGTATGA
TGCTGGAGCAACAGAAATTCCATAGTGATATCCAGGTAAATCTGGATTCT
TCTGCGGCTGGGACCGAGAGCGGAGGTCATGTAGTGAGCCAGTCTGAAAA
GTTCACCGAACGTAACCGCGAGATGAAACGCTAA

By “Tardigrade 107838 polypeptide” is meant a polypeptide or fragment thereof having at least about 85%, 86%, 87%, 88%, 89%, 90%, 91%, 92%, 93%, 94%, 95%, 96%, 97%, 98%, 99%, or 100% amino acid identity to NCBI Accession No. POCU51 and having intrinsically disordered protein properties, the polypeptide having the structure:

>Tardigrade_107838
(SEQ ID NO: 16)
MSAEAMNMNMNQDAVFIPPPEGEQYERKEKQEIQQTSYLQSQVKVPLVN
LPAPFFSTSFSAQEILGEGFQASISRISAVSEELSSIEIPELAEEARRD
FAAKTREQEMLSANYQKEVERKTEAYRKQQEVEADKIRKELEKQHLRDV
EFRKDIVEMAIENQKKMIDVESRYAKKDMDRERVKVRMMLEQQKFHSDI
QVNLDSSAAGTETGGQVVSESQKFTERNRQIKQ

By “Tardigrade 107838 nucleic acid molecule” is meant a polynucleotide encoding a Tardigrade 107838 polypeptide. An exemplary endogenous Tardigrade 107838 nucleic acid molecule is:

>endogenous_Tardigrade_107838_nucleic_acid
(SEQ ID NO: 17)
ATGTCTGCTGAAGCGATGAACATGAACATGAACCAGGACGCCGTGTTTA
TTCCCCCGCCCGAGGGTGAGCAGTACGAGCGAAAGGAGAAGCAGGAGAT
CCAGCAGACCAGCTACCTGCAGTCCCAGGTCAAGGTGCCCCTTGTCAAC
CTCCCCGCTCCGTTCTTCAGCACTTCCTTTTCTGCCCAAGAAATTCTCG
GCGAAGGTTTCCAGGCTTCGATTTCGCGCATCAGCGCCGTCTCGGAAGA
GCTGTCGTCCATCGAGATTCCCGAACTGGCCGAAGAGGCCCGTCGCGAC
TTCGCTGCCAAAACCCGTGAGCAGGAGATGCTGTCAGCCAATTATCAGA
AGGAAGTGGAGCGCAAGACCGAGGCCTACCGCAAGCAGCAGGAAGTCGA
GGCCGACAAGATCCGCAAAGAACTGGAGAAGCAGCATCTGCGTGACGTT
GAGTTCCGCAAGGACATTGTCGAGATGGCCATCGAGAACCAGAAGAAAA
TGATCGACGTGGAGAGCCGCTACGCCAAGAAGGACATGGACCGCGAACG
CGTCAAGGTTCGAATGATGCTCGAGCAGCAAAAGTTCCACAGCGACATC
CAGGTCAATCTGGATTCTTCGGCTGCTGGCACGGAAACTGGAGGTCAGG
TGGTGTCGGAATCTCAAAAGTTCACCGAACGAAACCGCCAGATAAAGCA
ATAA

By “Tardigrade 107838 nucleic acid molecule” is meant a polynucleotide encoding a Tardigrade 107838 polypeptide. An exemplary recombinant Tardigrade 107838 nucleic acid molecule is:

>recombinant_Tardigrade_107838_nucleic_acid
(SEQ ID NO: 18)
ATGTCAGCGGAAGCTATGAACATGAACATGAACCAGGATGCAGTTTTTAT
TCCTCCTCCGGAAGGCGAACAGTATGAACGCAAAGAGAAACAAGAGATTC
AACAGACGTCGTACCTGCAGAGCCAGGTAAAGGTTCCGCTGGTCAACCTG
CCCGCACCTTTCTTCTCAACCTCCTTCTCCGCGCAGGAGATCCTTGGGGA
AGGTTTCCAGGCTAGTATTTCTCGGATTTCCGCCGTTAGTGAAGAACTGT
CCTCTATCGAAATTCCGGAGCTGGCGGAAGAGGCCCGTCGTGACTTTGCG
GCCAAAACGCGCGAACAGGAGATGCTGTCTGCTAACTATCAAAAAGAAGT
TGAGCGCAAAACTGAAGCCTATCGGAAGCAGCAAGAAGTGGAGGCAGACA
AAATTCGGAAAGAGCTGGAAAAGCAGCATCTGCGTGACGTGGAGTTCCGC
AAAGATATTGTAGAGATGGCAATCGAAAACCAGAAAAAAATGATTGATGT
CGAAAGCCGTTATGCAAAAAAAGATATGGACCGCGAACGTGTGAAAGTCC
GTATGATGCTCGAGCAGCAAAAATTTCATAGCGACATCCAGGTAAATCTC
GATTCTAGCGCTGCAGGGACCGAAACTGGAGGCCAAGTTGTTTCAGAATC
TCAGAAATTTACCGAACGTAATCGTCAGATTAAACAGTGA

It is expressly contemplated that the compositions and methods of the instant disclosure can include any of a number of IDPs that possess cryoprotective and/or vitrification properties. Such IDPs are contemplated to include not only tardigrade-specific IDPs (TSIDPs) in addition to the above-listed TSIDPs, but also IDPs derived from other organisms, where such non-tardigrade IDPs possess cryoprotective and/or vitrification properties similar to those identified herein for TSIDPs.

A “therapeutically effective amount” of an agent described herein (e.g., a recombinant probiotic bacteria) is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 is a diagram depicting a Tardigrade-inspired model to increase survival of freeze-dried probiotics as a result of expression of protective tardigrade-specific intrinsically disordered proteins according to an exemplary embodiment of the disclosure.

FIGS. 2A-2D show a micrograph and 3 bar charts, respectively, depicting viability results of modified BL21 E. coli expressing isolated tardigrade-specific intrinsically disordered proteins (TSIDPs) compared to untransformed control according to an exemplary embodiment of the disclosure. FIG. 2A is a micrograph of an exemplary sample tardigrade. FIG. 2B is a bar chart showing improved E. coli survival to slow freezing and desiccation in the presence of TSIDPS. FIG. 2C is a bar chart showing improved E. coli survival to snap freezing and desiccation. FIG. 2D is a bar chart showing absolute averaged survival improvement for five TSIDP genes (e.g., 77580, 89226, 94063, 106094, 107838) compared to BL21 control. Four-to-ten-fold improvements in survival percentage were observed in the engineered bacterium according to the exemplary embodiments shown.

### DETAILED DESCRIPTION OF THE DISCLOSURE

The present disclosure is based, at least in part, on the discovery that recombinant bacteria (e.g., probiotic bacteria) expressing one or more tardigrade-specific intrinsically disordered proteins (TSIDPs) such as, for example, TSIDPs 77580, 89226, 94063, 106094, and/or 107838 impart desiccation resistant characteristics onto the recombinant bacteria that greatly enhance viability after freeze-drying/lyophilization. Additionally, the present disclosure provides recombinant probiotic bacteria that may be used to enhance access, ease of use, and efficacy of probiotics used for the treatment of rare genetic diseases like urea cycle disorders (UCD) and phenylketonuria (PKU), as well as inflammatory bowel disease (IBD) and certain cancers.

Tardigrades (a.k.a., water bears or moss piglets) are resilient organisms capable of surviving a wide range of extreme stresses such as desiccation. Tardigrades are micro-animals with remarkable tolerance to various physical stressors, including starvation, dehydration, extreme temperature and pressure, radiation, and oxygen deprivation [1,2]. For example, tardigrades are able to survive complete desiccation for up to 10 years. There are over 1,000 known species of the phylum Tardigrada living on land, freshwater and at sea, many of which are recognized as some of the most resilient organisms in nature [3].

Increased understanding of tardigrade genetics and physiology has led to the identification of a range of tardigrade-specific intrinsically disordered proteins (TSIDPs) that are strongly associated with their tolerance to desiccation and radiation [4,5]. Intrinsically disordered proteins (IDP) are proteins that lack a fixed or ordered 3-dimensional structure. IDPs may cover a wide spectrum of structural states ranging from fully unstructured to partially structured, and may include tertiary motifs such as, for example, random coils, molten globules, and large multi-domains connected by flexible linkers. Interestingly, the addition of these proteins to heterologous systems such as yeast and purified enzyme suspensions appear to improve their stability after desiccation [4].

Natural and engineered probiotics are increasingly being used as living medicines for a variety of clinical applications [6,7]. Depending on the strain and the number of organisms present in a specific probiotic, different pharmaceutical formulations may be used to maximize the yield and efficacy of such products. Currently, probiotics are available in various formats, including liquid tablets, supplemented dairy products, and freeze-dried pills. From these, freeze-dried probiotics are among the most convenient and desirable presentations of these therapeutics. However, degradation processes in freeze-dried probiotics remain a barrier for their viability and functionality [8].

According to the techniques herein, the expression of TSIDPs may provide protection to diverse biological systems exposed to an array of physical stressors. In particular, engineering beneficial strains of probiotic bacteria to express one or more TSIDPs may impart tardigrade characteristics of resilience (e.g., desiccation resistance) to the probiotic bacteria. For example, the on-demand expression of TSIDPs may improve the stability of freeze-dried pharmaceutical products. Natural and engineered probiotics, for example, are important biological products with limited stability in freeze-dried formats, which may be improved according to the techniques herein by expressing TSIDPs within target strains of probiotic bacteria to enhance their viability during freeze-dried preservation. The present disclosure provides a general probiotic model using Escherichia coli BL21, and shows that transforming such probiotic bacteria with tardigrade-specific IDP genes results in 4-10 fold improvement in survival following freeze-drying.

According to the techniques herein, the expression of TSIDPs within a bacterial probiotic model may be used to enhance the ability of a probiotic (e.g. bacterial/microbial strain or strains) to survive freeze-drying, thereby increasing the robustness of the probiotic and eliminating the need for refrigeration for its distribution and storage. As described in detail below, an engineered probiotic model using E. coli BL21 was modified to express five independent TSIDPs (e.g. 77580, 89226, 94063, 106094, and 107838) as a means to improve survival of probiotic colonies following freeze-drying. The expression of TSIDPs was tested in conjunction with a 10% sucrose cryoprotectant and found to improve overall viability of freeze-dried E. coli after rehydration. Survival improvements for this TSIDP-enhanced probiotic model ranged from 4- to 10-fold relative to controls. The present disclosure provides that tardigrade-bioinspired preservation strategies for freeze-dried probiotics may improve the robustness and clinical efficacy of living medicines (e.g., probiotics).

Tardigrade proteins 77580, 89226, 94063, 106094, and/or 107838 have been poorly characterized in the art, but are known to act as molecular shields in water-deficient conditions. These tardigrade proteins are important for desiccation tolerance by forming non-crystalline amorphous solids upon desiccation, and this vitrified state mirrors their protective capabilities.

An “effective amount” is an amount (e.g., of one or more probiotic bacteria) sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

### Combination Treatments

In the compositions and methods of the present disclosure, the probiotic may be any probiotic species, such as, for example, a probiotic selected from the group consisting of Lactobacillus acidophilus, L. bulgaricus, L. casei, L. paracasei, L. fermentum, L. plantarum, L. rhamnosus, L. salivarius, Bifidobacterium bifidum, B. infantis, B. animalis subsp. lactis, B. longum, Streptococcus thermophilis, Enterococcus faecalis, and E. faecium. Combinations of probiotic species are also contemplated under the compositions and methods of the present disclosure such as for example, a combination of L. acidophilus and B. animalis subsp. lactis. In certain embodiments, the concentration of probiotics contemplated under the composition and method of the present disclosure is in a range of approximately 10⁶ to approximately 10¹⁰ colonies per dosage form; this concentration is an individual concentration range for each probiotic present in the composition.

In certain embodiments, the probiotic compositions of the instant disclosure can optionally be combined with other modalities, especially where therapeutic preparations are contemplated. For instance, exemplary art-recognized treatments for IBD include but are not limited to: anti-inflammatories including corticosteroids and aminosalicylates, such as mesalamine, balsalazide and olsalazine; immunosuppressant drugs including azathioprine, mercaptopurine, cyclosporine and methotrexate; TNF-α inhibitors including infliximab, adalimumab and golimumab, as well as other biologic therapies including natalizumab, vedolizumab and ustekinumab; antibiotics where infection is a concern, including ciprofloxacin and metronidazole; and anti-diarrheal medications, including fiber supplements such as psyllium powder or methylcellulose, as well as drugs such as loperamide. Art-recognized treatments for urea cycle disorders (UCDs) include the ammonia scavengers sodium phenylbutyrate and buphenyl. Art-recognized treatments for PKU include PKU formula, large neutral amino acid supplementation and/or enzyme replacement therapy. Art-recognized cancer treatments include resection, radiation, immunotherapies, chemotherapeutics, etc. It is therefore expressly contemplated that the probiotic compositions of the instant disclosure can be combined with any number of additional therapeutics, e.g., as co-administered agents, as agents that are combined within a single formulation, or as agents that are separately administered to a subject over the same, roughly defined period of time (e.g., over roughly the same period of hours, days, weeks and/or months).

Administration of a composition of the present disclosure to a subject will follow general protocols for the administration described herein, and the general protocols for the administration of a particular secondary therapy will also be followed, considering the toxicity, if any, of the treatment. It is expected that the treatment cycles would be repeated as necessary. It also is contemplated that various standard therapies may be applied in combination with the described therapies.

### Pharmaceutical Compositions

Agents of the present disclosure can be incorporated into a variety of formulations for therapeutic use (e.g., by administration of encapsulated freeze-dried or lyophilized probiotics) or in the manufacture of a medicament (e.g., for treating or preventing urea cycle disorders (UCD) and phenylketonuria (PKU), as well as inflammatory bowel disease (IBD) and certain cancers) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, powders, and chewing gum, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977): 1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

### Formulations and Dosage

As also noted elsewhere herein, the probiotic compositions of the present disclosure may be formulated into oral dosage forms, such as tablets, caplets, or capsules. The probiotic tablets and caplets of the present disclosure may be formulated for swallowing or for chewing. In the latter case, the tablets and caplets can be prepared with flavoring. Where necessary, the flavored chewable tablets and caplets may include a sweetener, which may be an artificial or natural sweetener or both. The probiotic capsules of the present disclosure will be manufactured primarily for swallowing.

Procedures for preparing tablets, caplets, and capsules are known to those of ordinary skill in the art and include without limitation wet granulation, dry granulation, and direct compression (for tablets and caplets).

Wet and dry granulation is used to manufacture tablets, caplets, or capsules. With granulation techniques, a chilsonation can be used to manufacture the powder for the dosage forms. A chilsonator houses grooved, rotating rollers that are pressed tightly against one another by hydraulic pressure. Raw materials are placed into the hopper of the chilsonator and are fed by a system of horizontal and vertical screws into the rollers. As materials pass through the grooves in the rollers, it is compacted under very high pressure and emerges from the chilsonator as dense sheets. The sheets are milled into a fine granular powder using a Fitz mill and then passed through a screen to produce a uniform free flowing granule. The chilsonation process results in a finished powder that is two to four times denser than the starting material, a feature that permits the ingredients to be fashioned into the desired dosage form.

With dry granulation, the powder may be incorporated into a gelatin capsule or it may be mixed with gelatin to form a tablet or caplet. With wet granulation, the powder is moistened thus creating large “chunks” of material that are subsequently dried and milled to convert the chunks to particles of a desired size for the manufacturing process. Once the particles of a desired size are obtained, the particles are incorporated into a gelatin capsule or mixed with gelatin to form a tablet or caplet.

Most direct compression formulations consist of three types of ingredients: an inert carrier that provides volume to the final dosage form; a lubricant that aids in the compression process; and the active ingredients. Carriers may be present in dosage forms in many ranges, from 0.5% w/w to 95% w/w. Within the context of the present disclosure, carriers used to formulate the probiotic compositions of the present disclosure will generally be in the range of approximately 30% w/w to approximately 98% w/w.

With direct compression, the ingredients are mixed in a batch blender, such as a twin-shell or V-blender, and discharged into a bin (usually portable), which then feeds a chute to the tableting process. As an alternative to batch blending, in-bin blending is also used. With in-bin blending, the unmixed material is placed in a portable bin, which may contain internal baffles, and is tumbled; in-bin blending avoids the need to transfer the material from the blender to the portable container. In direct compression formulations, there is a wide particle-size distribution with the active agent usually being at the fine end of the range. To avoid segregation of the particles, reliable flow must be maintained in the bins at all time. To ensure that all tablets are of the highest quality, tablets must be sampled at regular intervals during production for active ingredient concentration, tablet hardness, and dissolution rates.

Direct compression may be used to make tablets and caplets for swallowing and also chewable tablets and caplets. With the latter, the hardness of the tablets and caplets will need to be reduced and the ingredients of the tablets and caplets will need to be adjusted to ensure that the tablets and caplets have a pleasant taste and pleasant mouth-feel. The taste of the chewable tablets and caplets may be adjusted through the use of various flavorings and sweeteners and the mouth-feel of the chewable tablets and caplets may be adjusted though the use of varying prebiotics, such as inulin, lactitol, mannitol, etc. The mouth feel of the chewable tablets must be adjusted to ensure that the when the tablet or caplet body is crushed, it folds into the flavored matrix; if the body is too brittle, it may fracture causing an uncomfortable sensation in the user's mouth. For chewable tablets, where taste is significant to user acceptability, the product should also have a pleasant odor when the bulk package is opened. Each of the features of the chewable tablet may be attained by adjusting the excipients until the desired properties are achieved.

Depending on the final size and characteristics of the tablets and caplets, i.e., size, hardness, brittleness, mouth feel for chewing, etc., two different direct compression machines may be used to manufacture the dosage forms. Single punch presses typically exhibit low compression speeds while rotary presses exhibit high compression speeds.

The probiotic compositions of the present disclosure may also be incorporated into chewing gum. The chewing gum preferably includes a flavor and may be hard chewing gum or soft chewing gum. Where necessary, the flavored hard or soft chewing gum may include a sweetener, which may be an artificial or natural sweetener or both. Procedures for manufacturing gum are known to those of ordinary skill in the art.

Gum is traditionally made using four ingredients: a gum base, such as a resin obtained from pine trees; a natural sweetener, such as sugar, or an artificial sweetener; a softener, such as glycerin; and flavoring. The raw materials for the gum are mixed with a mixer while kept at a constant temperature during the prescribed processing time. Mixed gum materials are then sent to a hopper and extruded by twin screws to make a gum sheet. The inside and outside of the gum sheets are powdered to prevent sticking to the matching during rolling and packaging. During the extruding process, the gum sheet thickness is controlled with a roller. After extrusion, the gum sheet is scored and cut for proper sizing, passed through a cooling tunnel, and stacked on trays for packaging. Where the gum is a hard chewing gum, such as a chewing gum tablet, the gum is coated between the cutting and cooling steps. Specially designed machines are available for each of the steps; thus, gum can be made using a mixing machine, an extruding machine, a forming machine, a cooling machine, and a stacking machine.

Gum may also be prepared using the direct compression procedure described in U.S. Patent Publication No. 2004/0013767 to Norman and Amin, which is incorporated by reference herein. Under this procedure, a gum base, granulating agent, processing aid, and one or more lubricants are mixed and subjected to direct compression on a traditional tabletting machine. Sweeteners, colorings, and flavorings may also be added to the mixture. A commercially available mixture of polyol(s) and/or sugars in a gum base is sold commercially as PHARMAGUM® (SPI Polyols, Inc., New Castle, Del.). The direct compression procedure is particularly useful for preparing chewing gum tablets.

In both of the foregoing procedures, the probiotic can be added during the initial mixing stage prior to extrusion or direct compression.

In addition to the foregoing, the probiotic compositions of the present disclosure may be prepared as a powder that is intended to be dissolved in a liquid, such as water, milk, juice, and yogurt. It is understood that the individual liquids may be mixed together where appropriate. For example, the probiotic formulation may be combined with fruit juice and yogurt or milk and yogurt to make probiotic yogurt shakes. The probiotic formulation may also be combined with milk and ice cream to make probiotic milk shakes. Flavorings for the probiotic liquid formulations contemplated under the disclosure are known to those of ordinary skill in the art.

Examples of excipients that may be used to formulate appropriate dosage forms include binders, disintegrants, lubricants, coatings, plasticizers, compression agents, wet granulation agents, and sweeteners, all of which are known to those of ordinary skill in the art to which the disclosure pertains. All of the following examples are provided by way of illustration and not limitation. Binders are used where appropriate to help the dosage form ingredients still together. Examples of binders include carbopol, povidone, and xanthan gum. Lubricants are generally always used in the manufacture of dosage forms by direct compression in order to prevent the compacted powder mass from sticking to the equipment during the tabletting or encapsulation process. Examples of lubricants include calcium stearate, magnesium stearate, stearic acid, sodium stearyl fumerate, and vegetable based fatty acids. Disintegrants aid in the break up of the compacted mass when placed in a fluid environment. Examples of disintegrants include sodium croscarmellose, crospovidone, gellan gum, hydroxypropyl cellulose, starch, and sodium starch glycolate. Coatings are used to control the solubility of the drug. Examples of coatings include carrageenan, cellulose acetate phthalate, ethylcelulose, gellan gum, matodextrin, methacrylates, methylcellulose, microcrystalline cellulose, and shellac. Plasticizers are used to control the release rate of the drug from the dosage form. Examples of plasticizers include citrate esters, dibutyl sebacate, diethyl phthalate, polyvinylacetate phthalate, and triacetin. Compression agents include calcium carbonate, dextrose, fructose, guar gum, honey, lactose, maltodextrin, maltose, mannitol, microcrystalline cellulose, molasses, sorbitol, starch, and sucrose. Wet granulation agents include calcium carbonate, lactose, maltodextrin, mannitol, microcrystalline cellulose, povidone, and starch. Sweeteners include aspartame, dextrose, fructose, honey, lactose, maltodextrin, maltose, mannitol, molasses, monoammonium glycyrrhizinate, sorbitol, sucralose, and sucrose. Excipients that are generally used in the manufacture of chewable tablets include by way of illustration and not limitation, dextrose, fructose, guar gum, lactose, maltodextrin, maltose, mannitol, microcrystalline cellulose, and sorbitol. As is evident from the foregoing list, many of the same ingredients may be used for various different purposes in various different dosage forms.

In certain probiotic compositions described herein, the probiotic can be present in a range of approximately 10⁶ colonies per dosage unit to approximately 10¹⁰ colonies per dosage unit, although lower (e.g., 10 colonies per dosage unit, 100 colonies per dosage unit, 1000 colonies per dosage unit, 10,000 colonies per dosage unit, 100,000 colonies per dosage unit) or higher counts (e.g., 10¹¹ colonies per dosage unit, 10¹² colonies per dosage unit, 10¹³ colonies per dosage unit, 10¹⁴ colonies per dosage unit, 10¹⁵ colonies per dosage unit, 10¹⁶ colonies per dosage unit, 10¹⁷ colonies per dosage unit, 10¹⁸ colonies per dosage unit) are also acceptable. Where the probiotic composition is to be administered to an adult human being, i.e., a human being over the age of 16, the probiotic is preferably in the range of at least 10⁹, more preferably 10¹⁰ or even higher. Where the probiotic composition is to be administered to a child, i.e., a human being under the age of 16, the probiotic is preferably in the range of approximately 10⁶ to approximately 10⁸. Routine studies can be performed to determine the conditions at which the probiotic compositions of the present disclosure have the longest duration. Storage conditions (e.g., frozen, 4° C., room temperature, high temperature storage, etc.) are expected to play a role in the net activity and duration of the probiotic compositions, even in view of the stabilizing proteins present in the preparations of the instant disclosure. Further, the presence of a desiccant in the storage package may also make a difference under certain storage conditions, although, this is not always the case. Examples of desiccants that can be used with the present disclosure include without limitation, silica gel (silicon dioxide), indicating silica gel (silica gel washed with cobalt chloride), montmorillonite clay, calcium oxide, calcium sulfate, activated alumina beads, and molecular sieve (e.g., aluminosilicate materials or synthetic compounds such as clays, porous glass, microporous charcoal, or active carbon).

The probiotic compositions of the present disclosure have widespread utility in the manufacture of various consumable commercial products that upon ingestion by a human or an animal will regulate their digestive systems by ensuring that the digestive tract is populated by live probiotic species. Because it is essential that probiotic species are viable upon ingestion, the enhanced stability of the probiotic species in the probiotic compositions of the present disclosure ensure that the probiotic compositions have the capability to reduce the number of pathogenic species in the colon. Examples of pathogenic bacteria that may be reduced through ingestion of the probiotic compositions of the present disclosure include without limitation, Campylobacter jejuni, E. coli, S.s aureaus, Vibrio cholera, bacteroides, clostridia, klebsiella, listeria, proteus, salmonella, shigella, and veilloniella. An example of pathogenic yeast that may be reduced though ingestion of the probiotic compositions of the present disclosure include without limitation C. albicans. As previously noted, beneficial probiotic species, such as lactobacilli and bifidobacteria keep those potentially disease-causing pathogens under control preventing disease-related dysfunctions.

An advantage of the probiotic formulations of the presenting disclosure is that they are robustly stable, retaining activity when freeze-dried and/or lyophilized, optionally over extended durations of time and/or harsh environmental conditions. In certain embodiments, the probiotic formulations of the present disclosure are stable at room temperature and do not need special conditions in order to improve or sustain the shelf life of the product.

A composition, preparation or other therapy of the instant disclosure is administered to the subject in an amount sufficient to achieve a desired effect at a desired site (e.g., amelioration of gastrointestinal tract health, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., encapsulated freeze-dried or lyophilized probiotics) described herein. In certain embodiments, a dose described herein includes independently between 1 mg and 3 mg, inclusive, of an agent (e.g., encapsulated freeze-dried or lyophilized probiotics) described herein. In certain embodiments, a dose described herein includes independently between 3 mg and 10 mg, inclusive, of an agent (e.g., encapsulated freeze-dried or lyophilized probiotics) described herein. In certain embodiments, a dose described herein includes independently between 10 mg and 30 mg, inclusive, of an agent (e.g., encapsulated freeze-dried or lyophilized probiotics) described herein. In certain embodiments, a dose described herein includes independently between 30 mg and 100 mg, inclusive, of an agent (e.g., encapsulated freeze-dried or lyophilized probiotics) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., encapsulated freeze-dried or lyophilized probiotics) or composition, as described herein, can be administered in combination with one or more additional pharmaceutical agents (e.g., therapeutically and/or prophylactically active agents), which are different from the agent or composition and may be useful as, e.g., combination therapies.

The agents or compositions can be administered in combination with additional pharmaceutical agents that improve their activity (e.g., activity (e.g., potency and/or efficacy) in treating a disease (e.g., IBD) in a subject in need thereof, in preventing a disease in a subject in need thereof, in reducing the risk of developing a disease in a subject in need thereof, etc. in a subject or cell. In certain embodiments, a pharmaceutical composition described herein including an agent (e.g., encapsulated freeze-dried or lyophilized probiotics) described herein and an additional pharmaceutical agent shows a synergistic effect that is absent in a pharmaceutical composition including one of the agents and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an immunotherapy, an antioxidant, an anti-inflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will consider compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, additional probiotic preparations, anti-cancer agents, immunotherapy and/or immunomodulatory agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising freeze-dried/lyophilized reagents (e.g., recombinant probiotic bacteria expressing one or more tardigrade-specific intrinsically disordered proteins) of this disclosure. In some embodiments, the kits further include instructions for use in accordance with the methods of this disclosure. In some embodiments, these instructions comprise a description of administration of the reagents according to any of the methods of this disclosure. The kit may further comprise a description of selecting an individual suitable for treatment with the probiotics described herein based on identifying whether that subject has a disease or condition selected from the group consisting of urea cycle disorders (UCD) and phenylketonuria (PKU), as well as inflammatory bowel disease (IBD) and certain cancers.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like. In certain embodiments, at least one active agent in the composition is a recombinant probiotic bacterium expressing one or more TSIDPs or a probiotic bacterium formulated in combination with one or more TSIDPs. The container may further comprise a second pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory uses of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

Competent E. coli BL21 transformation for expression of tardigrade-specific TSIDP proteins was performed based on a modified protocol from Boothby et al. [4]. For example, codon-optimized gBlocks encoding tardigrade-specific intrinsically disordered proteins (TSIDPs) (e.g., 77580, 89226, 94063, 106094, 107838) were synthesized (Integrated DNA Technologies) and cloned into the pET28b expression vector. BL21star (DE3) E. coli were transformed with pET28b+CAHS plasmids. A single bacterial colony was used to inoculate 10 mL of Lennox broth (LB, 10 g/L tryptone, 5 g/L yeast extract, 5 g/L NaCl) supplemented with 60 μg/mL of kanamycin. The culture was shaken at 37° C. overnight (New Brunswick Scientific Innova 126, 225 rpm). Three of these cultures were used to inoculate fresh LB medium at a dilution of 1/10,000. Cultures were shaken at 37° C. until reaching stationary phase. Cells were then sub-cultured into fresh LB media until an optical density at 600 nm reached 0.1. Transformed cell cultures for each exogenous TSIDP gene group were divided into pairs to receive IPTG (1 mM final concentration) to induce expression or as not expressing controls. After 3 hours, the cells were plated or washed in lyophilization solution. Cells were then frozen at −20° C. for 30 minutes or flash frozen in liquid nitrogen. Cell pellets were subjected to lyophilization overnight and then plated.

### Example 2: TSIDPs Impart Cryoprotective Activity to E. coli

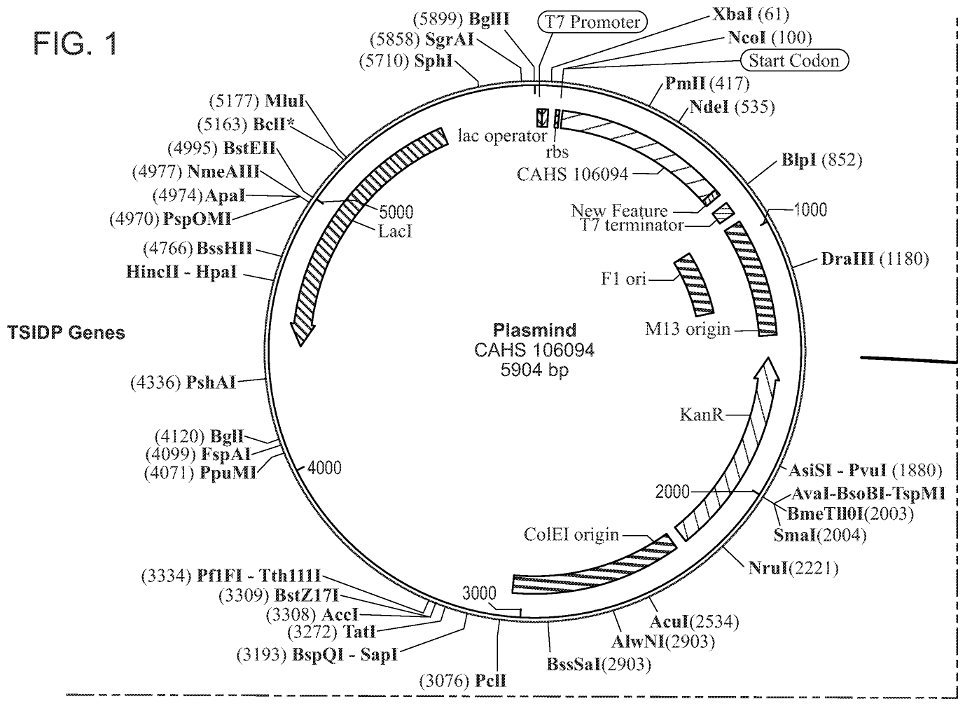

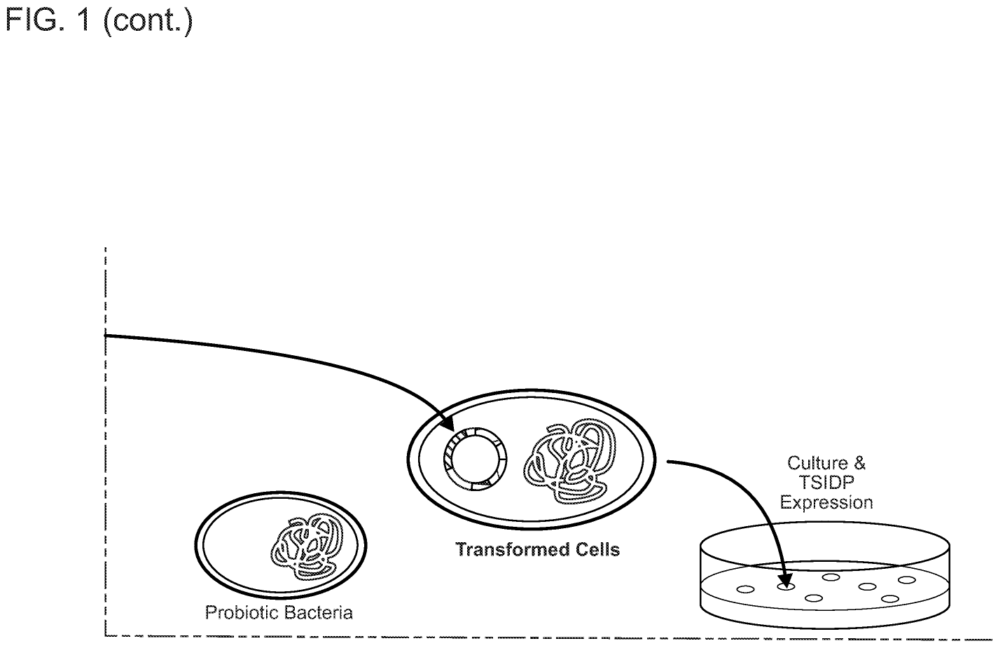

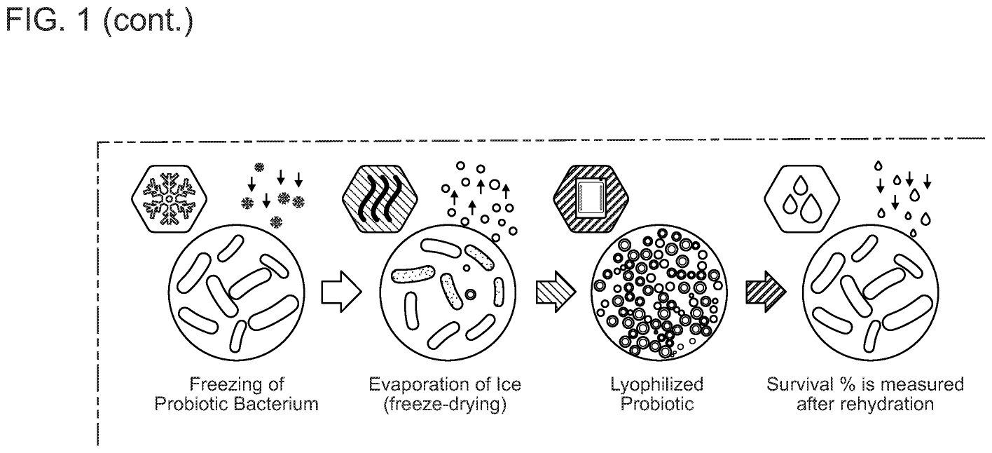

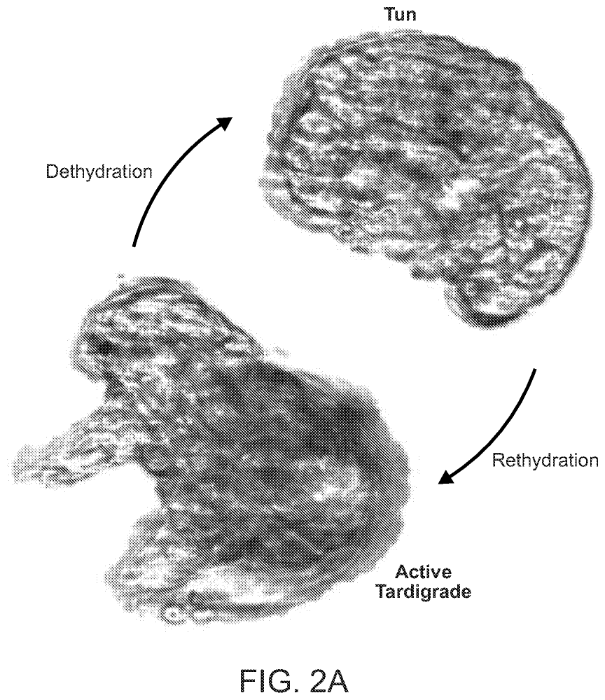

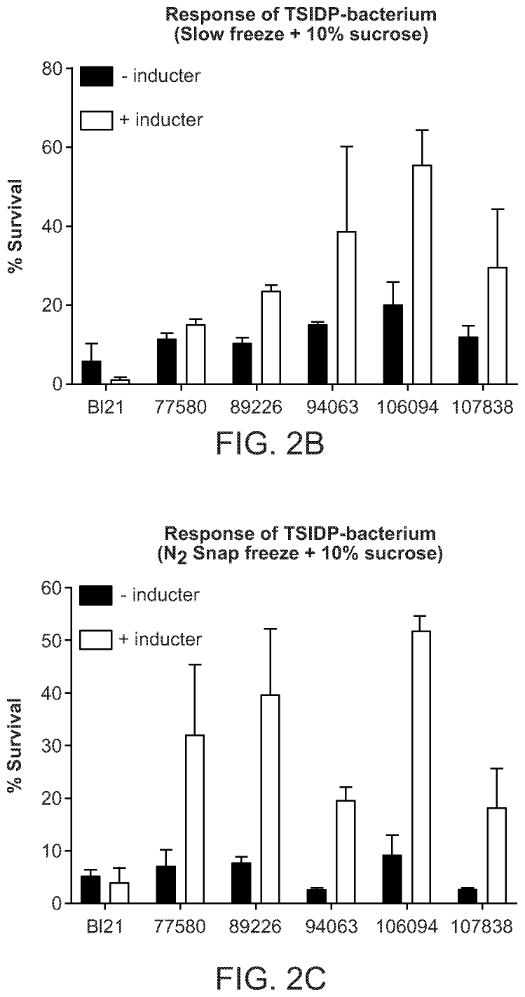

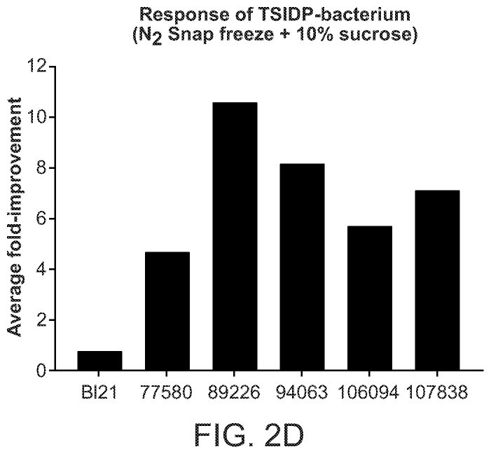

The viability of freeze-dried engineered probiotics used as living therapeutics can be improved by the expression of tardigrade-specific intrinsically disordered proteins (TSIDPs). TSIDPs are naturally occurring proteins expressed in tardigrades (FIGS. 2A-2D), which can be transferred to other organisms by simple transformation processes using a reduced number of foreign genes (FIG. 1). The expression of TSIDPs were tested in conjunction with 10% sucrose as a commonly used cryoprotectant to improve overall viability. E. coli expressing each of five tested TSIDPs that were frozen and dried in a cryoprotective solution containing 10% sucrose showed survival improvement (ranging from 4-10 fold enhancement) in the event of freeze-drying (FIGS. 2B, 2C and 2D). Best achieved averaged protection within expressing and non-expressing transformed bacterium was seen using TSIDP gene 88226, whereas the highest average survival percentage was seen in bacteria transformed with TSIDP gene 106094 (FIGS. 2C and 2D).

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

In addition, where features or aspects of the invention are described in terms of Markush groups or other grouping of alternatives, those skilled in the art will recognize that the invention is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this invention are described herein, including the best mode known to the inventors for carrying out the invention. Variations of those embodiments may become apparent to those of ordinary skill in the art upon reading the foregoing description.

The invention illustratively described herein suitably can be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this invention as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

### REFERENCES

1. Sørensen-Hygum, T. L., Stuart, R. M., Jørgensen, A. and Møbjerg, N., 2018. Modelling extreme desiccation tolerance in a marine tardigrade. Scientific reports, 8(1), p. 11495.
2. Sloan, D., Batista, R. A. and Loeb, A., 2017. The resilience of life to astrophysical events. Scientific reports, 7(1), p. 5419.
3. Nichols, P. B., 2005. Tardigrade evolution and ecology.
4. Boothby, T. C., Tapia, H., Brozena, A. H., Piszkiewicz, S., Smith, A. E., Giovannini, I., Rebecchi, L., Pielak, G. J., Koshland, D. and Goldstein, B., 2017. Tardigrades use intrinsically disordered proteins to survive desiccation. Molecular cell, 65(6), pp. 975-984.
5. Hashimoto, T., Horikawa, D. D., Saito, Y., Kuwahara, H., Kozuka-Hata, H., Shin, T., Minakuchi, Y., Ohishi, K., Motoyama, A., Aizu, T. and Enomoto, A., 2016. Extremotolerant tardigrade genome and improved radiotolerance of human cultured cells by tardigrade-unique protein. Nature communications, 7, p. 12808.
6. Pandey, K. R., Naik, S. R. and Vakil, B. V., 2015. Probiotics, prebiotics and synbiotics-a review. Journal of food science and technology, 52(12), pp. 7577-7587.
7. Mao, N., Cubillos-Ruiz, A., Cameron, D. E. and Collins, J. J., 2018. Probiotic strains detect and suppress cholera in mice. Science translational medicine, 10(445), p.eaao2586.
8. Iaconelli, C., Lemetais, G., Kechaou, N., Chain, F., Bermndez-Humarin, L. G., Langella, P., Gervais, P. and Beney, L., 2015. Drying process strongly affects probiotics viability and functionalities. Journal of biotechnology, 214, pp. 17-26.

1. A composition comprising a probiotic bacteria population and one or more tardigrade-specific intrinsically disordered proteins (TSIDPs)

2. The composition of claim 1, wherein bacteria of the probiotic bacteria population express the one or more TSIDPs.

3. The composition of claim 1, wherein the probiotic bacteria population comprises bacteria of one or more species selected from the genera consisting of Lactobacillus, Bifidobacterium, Enterococcus, Streptococcus, Pediococcus, Leuconostoc, Bacillus, Escherichia, and Saccharomyces.

4. The composition of claim 3, wherein the one or more probiotic bacterial species comprise a Lactobacillus or Bifidobacterium species.

5. The composition of claim 1, wherein the TSIDPs are selected from the group consisting of tardigrade protein 77580, tardigrade protein 89226, tardigrade protein 94063, tardigrade protein 106094, tardigrade protein 107838, and combinations thereof.

6. The composition of claim 1, wherein the probiotic bacteria population and the one or more TSIDPs are present in an admixture.

7. A composition selected from the group consisting of:
A preparation for oral delivery to a subject, the preparation comprising one or more recombinant probiotic bacteria that express one or more tardigrade-specific intrinsically disordered proteins (TSIDPs);
A preparation for oral delivery possessing freeze-dried recombinant probiotic bacteria;
A freeze-dried preparation for oral delivery comprising one or more isolated tardigrade-specific intrinsically disordered protein (TSIDPs) and one or more isolated populations of probiotic bacteria;
A pharmaceutical composition comprising an isolated tardigrade-specific intrinsically disordered protein (TSIDP), an isolated population of probiotic bacteria, and a pharmaceutically acceptable carrier;
A pharmaceutical composition comprising recombinant probiotic bacteria that express a tardigrade-specific intrinsically disordered protein (TSIDP), and a pharmaceutically acceptable carrier;
A composition comprising a probiotic bacteria population and one or more natural or synthetic intrinsically disordered proteins (IDPs) that possesses cryoprotective or vitrification properties;
A preparation for oral delivery to a subject, the preparation comprising one or more recombinant probiotic bacteria that express one or more intrinsically disordered proteins (IDPs);
A preparation for oral delivery comprising freeze-dried recombinant probiotic bacteria;
A freeze-dried preparation for oral delivery comprising one or more isolated intrinsically disordered protein (IDPs) and one or more isolated populations of probiotic bacteria;
A pharmaceutical composition comprising an isolated intrinsically disordered protein (IDP), an isolated population of probiotic bacteria, and a pharmaceutically acceptable carrier; and
A pharmaceutical composition comprising recombinant probiotic bacteria that express an intrinsically disordered protein (IDP), and a pharmaceutically acceptable carrier.

8. The composition of claim 7, wherein the one or more recombinant probiotic bacteria express the one or more TSIDPs.

9. The composition of claim 7, wherein the one or more recombinant probiotic bacteria are of species selected from the genera consisting of Lactobacillus, Bifidobacterium, Enterococcus, Streptococcus, Pediococcus, Leuconostoc, Bacillus, Escherichia, and Saccharomyces.

10. The composition of claim 9, wherein the one or more probiotic bacterial species comprise a Lactobacillus or Bifidobacterium species.

11. The composition of claim 9, wherein the TSIDPs are selected from the group consisting of tardigrade protein 77580, tardigrade protein 89226, tardigrade protein 94063, tardigrade protein 106094, tardigrade protein 107838, and combinations thereof.

12. (canceled)

13. The composition of claim 7, wherein the recombinant probiotic bacteria express one or more TSIDPs selected from the group consisting of tardigrade protein 77580, tardigrade protein 89226, tardigrade protein 94063, tardigrade protein 106094, tardigrade protein 107838, and combinations thereof.

14. The composition of claim 7, wherein the recombinant probiotic bacteria express one or more IDPs selected from the group consisting of prokaryotic and eukaryotic proteins that possess cryoprotective or vitrification properties, and combinations thereof.

15. The composition of claim 7, wherein the one or more isolated populations of probiotic bacteria include species selected from the genera consisting of Lactobacillus, Bifidobacterium, Enterococcus, Streptococcus, Pediococcus, Leuconostoc, Bacillus, Escherichia, and Saccharomyces.

16. The composition of claim 7, wherein:
bacteria of the probiotic bacteria population express the one or more IDPs;
the probiotic bacteria population comprises bacteria of one or more species selected from the genera consisting of Lactobacillus, Bifidobacterium, Enterococcus, Streptococcus, Pediococcus, Leuconostoc, Bacillus, Escherichia, and Saccharomyces, optionally wherein the one or more probiotic bacterial species comprise a Lactobacillus or Bifidobacterium species;
the IDPs are selected from the group consisting of prokarvotic or eukarvotic proteins that possess cryoprotective or vitrification properties; and/or
the probiotic bacteria population and the one or more IDPs are present in an admixture.

17. The composition of claim 7, wherein:
the one or more IDPs possess cryoprotective or vitrification properties; and/or
the one or more recombinant probiotic bacteria are of species selected from the genera consisting of Lactobacillus, Bifidobacterium, Enterococcus, Streptococcus, Pediococcus, Leuconostoc, Bacillus, Escherichia, and Saccharomyces, optionally wherein the one or more probiotic bacterial species comprise a Lactobacillus or Bifidobacterium species and/or the IDPs are selected from the group consisting of prokaryotic or eukaryotic proteins that possess cryoprotective or vitrification properties, and combinations thereof.

18. A method selected from the group consisting of:
A method for treating or preventing a probiotic-treatable disease or disorder in a subject having or at risk of developing the probiotic-treatable disease or disorder, the method comprising administering to the subject:
(a) a pharmaceutical composition comprising an isolated TSIDP, an isolated population of probiotic bacteria, and a pharmaceutically acceptable carrier, or
(b) a pharmaceutical composition comprising recombinant probiotic bacteria that express a TSIDP, and a pharmaceutically acceptable carrier; and
A method for treating or preventing a probiotic-treatable disease or disorder in a subject having or at risk of developing the probiotic-treatable disease or disorder, the method comprising administering to the subject:
(a) a pharmaceutical composition comprising an isolated IDP, an isolated population of probiotic bacteria, and a pharmaceutically acceptable carrier, or
(b) a pharmaceutical composition comprising recombinant probiotic bacteria that express an IDP, and a pharmaceutically acceptable carrier.

19. The method of claim 18, wherein the probiotic-treatable disease or disorder is selected from the group consisting of a urea cycle disorder (UCD), phenylketonuria (PKU), inflammatory bowel disease (IBD) and a cancer.

20-38. (canceled)
